(19) United States
(12) Patent Application Publication
(10) Pub. No.: US 2012/0315265 A1
(43) Pub. Date: Dec. 13, 2012

(54) HYDROGEL-FORMING POLYMER, AND PREPARATION PROCESS AND USES THEREOF

(75) Inventors: Jui-Yang Lai, Tao-Yuan (TW); Ai-Ching Hsieh, Taipei City (TW); Pei-Lin Lu, New Taipei City (TW)

(73) Assignee: CHANG GUNG UNIVERSITY, Tao-Yuan (TW)

(21) Appl. No.: 13/354,467

(22) Filed: Jan. 20, 2012

(30) Foreign Application Priority Data
Jun. 13, 2011 (TW) 100120532

(51) Int. Cl. A61K 39/395; A61K 38/43; A61K 31/711; A61K 31/7105; A61K 31/713; A61K 35/76; A61K 38/02; A61K 31/7088; A61K 35/00; A61K 31/4178; A61P 29/00; A61P 23/00; A61P 35/00; A61P 31/00; A61P 31/12; A61P 37/02; A61P 27/02; A61P 27/06; A61P 27/12; A61P 27/14; C08H 1/00; A61P 27/08; A61K 47/42

(52) U.S. Cl. 424/130.1; 514/772.1; 424/94.1; 514/44 R; 514/44 A; 424/93.6; 514/1.1; 424/93.1; 514/397; 525/54.1

(57) ABSTRACT

Disclosed herein are a hydrogel-forming polymer and a process of making the same. Also disclosed are a sustained-release pharmaceutical composition that contains a therapeutic agent and the aforesaid hydrogel-forming polymer, and a method for treating or preventing an ophthalmic disorder, which includes intraocularly administering into an eye of a mammal in need of such treatment a sustained-release pharmaceutical composition that contains an ophthalmic drug and the aforesaid hydrogel-forming polymer.

### CROSS-REFERENCE TO RELATED APPLICATION

This application claims priority of Taiwanese Application No. 100120532, filed on Jun. 13, 2011.

### BACKGROUND OF THE INVENTION

1. Field of the Invention

This invention relates to a hydrogel-forming polymer that forms in situ a biodegradable thermo-responsive hydrogel in an aqueous medium having a physiological temperature. This invention also related to a process of making said hydrogel-forming polymer, in which a biodegradable component having amino functional groups is reacted with a thermo-responsive component having a carboxylic acid end group. This invention also relates to a sustained-release pharmaceutical composition comprising a therapeutic agent and said hydrogel-forming polymer as a carrier material. This invention also relates to a method for treating an eye disorder, in particular glaucoma and elevated intraocular pressure, comprising administering to an eye of a mammal in need of such treatment a sustained-release pharmaceutical composition comprising an ophthalmic drug and said hydrogel-forming polymer as a carrier material.

2. Description of the Related Art

Due to the uniqueness of eyes in terms of anatomic structure and physiology, delivering drugs into the eyes has long been a great challenge in the treatment of ophthalmic diseases. Currently, many of the ophthalmic preparations are manufactured in the form of eye drop which, however, has a severe problem of retaining the therapeutic agent(s) contained therein on the cornea for a sufficient time, as influenced by factors such as conjunctival blood flow, lymphatic clearance and tear dilution. Therefore, ophthalmic preparations of this type normally will be drained out of the eye within two minutes after topical administration. Said ophthalmic preparations also have difficulty getting into the interior of the eye due to the presence of tissue barriers such as cornea, sclera, retina, etc. As such, when administered via instillation, a very low amount of the ophthalmic preparations could reach the intraocular tissues. Because of poor ocular bioavailability, many ocular drugs are applied in high concentrations. This cause both ocular and systemic side-effects, which is often related to high peak drug concentrations in the eye and in systemic circulation. Further, the frequent periodic instillations of eye drops are necessary to maintain a continuous sustained therapeutic drug level. This gives the eye a massive and unpredictable dose of medication.

In order to enhance the bioavailability of ophthalmic drugs, various dosage forms of ophthalmic drugs have been developed, including ointments, nanoparticles, implantable drug depots, etc. However, oily viscous preparations for ophthalmic use (such as ointments) can cause blurred vision, matting of the eyelids, and may also be associated with discomfort by the patient as well as occasional ocular mucosal irritation. A drug depot encapsulating an ophthalmic drug can be surgically implanted into a patient's eye to provide a controlled release of said ophthalmic drug for a long duration of action. However, there exists a high risk to implant an ophthalmic drug depot since it is necessary to make an incision in the patient's eye and it requires a longer time for the patient to recover after eye operation, thus reducing patients' compliance.

Administering an ophthalmic drug into a patient's eye via injection can directly deliver said ophthalmic drug to a target site inside the patient's eye. This has the advantages of improving the bioavailability of said ophthalmic drug and reducing systemic side effects caused by over-dosing. However, repeated injection may be required when the ophthalmic drug has a short duration of action. As such, the patients have to suffer pain caused by repeated injection, which may further result in ocular injuries such as endophthalmitis, hemophthalmus, cataract, etc.

Worldwide, glaucoma is the second leading cause of blindness after cataract, and the number of people estimated to be bilaterally blind from glaucoma in 2020 will increase to 11.1 million (H. A. Quigley and A. T. Broman (2006), Br. J. Ophthalmol., 90:262-267). Eye drops are frequently used to administer medication for anti-glaucoma treatment. However, the main challenges with this type of dosage form include short precorneal residence time, poor corneal penetration, and low ocular bioavailability (Maria de la Fuente et al. (2010), Adv. Drug Deliv. Rev., 62(1):100-117). Hence, over the past few years, there has been an increased interest in the development of environmentally sensitive drug delivery systems (DDSs) for glaucoma therapy.

Given that the tear fluid contains electrolytes (i.e., Na⁺, K⁺, Ca²⁺) which may induce gel formation at physiological conditions, the vehicles made of known ion-sensitive polysaccharides such as deacetylated gellan gum (A. Rozier et al. (1989), Int. J. Pharm., 57(2):163-168) and alginate (Smadar Cohena et al. (1997), Journal of Controlled Release, 44(2):201-208) were found to improve the bioavailability of timolol in the cornea and aqueous humor of albino rabbits and to extend the duration of the intraocular pressure (IOP) reducing effect of pilocarpine to 10 hours, respectively.

Recently, Lin et al. reported that the pH-sensitive chitosan-poly(acrylic acid) nanoparticle suspension was better than simulated tear fluid and commercial eye drops at prolonging drug contact time and increasing pharmacological response during 6 hours of in vitro and in vivo pilocarpine release studies (Hong-Ru Lin et al. (2007), J. Biomater. Sci. Polymer Edn, 18(2):205-221). Nevertheless, for the pH triggered in situ gelling ophthalmic delivery system, it is highly recommended to neutralize the acidity of acrylic acid by incorporating the hydroxypropyl methylcellulose into the carrier matrix (B. Srividya et al. (2001), Journal of Controlled Release, 73(2-3):205-211).

Because their sol-gel phase transition can easily be modulated in the range from room temperature to body temperature, the thermo-sensitive polymers have also been extensively investigated as a vehicle for drug administration to the ocular surface. A study from Ging-Ho Hsiue et al. showed that after topical application of ophthalmic drops to rabbit eyes, the decreased pressure response of the formulation based on linear poly(N-isopropylacrylamide) (PNIPAAm) containing epinephrine lasted six-fold longer than that of the conventional eye drop (Ging-Ho Hsiue et al. (2002), Biomaterials, 23(2):457-462). In Biomaterials, 2003 June, 24(13):2423-2430, Ging-Ho Hsiue et al. further reported a thermo-sensitive ophthalmic drop for treating glaucoma. The eye drop, which was prepared by mixing linear poly(N-isopropylacrylamide-g-2-hydroxyethyl methacrylate) (PNIPAAm-g-PHEMA), PNIPAAm-g-PHEMA gel particles, and epinephrine (an anti-glaucoma drug), was a clear solution at room temperature and became a soft film after contacting the surface of cornea. The drug entrapped within the tangled polymer chains was released progressively after topical (subconjunctival) application. While said eye drop was shown to have an extended duration of action of 26 hours, the biodegradability of the thermo-sensitive polymers contained therein was not known.

Despite the aforementioned animal studies reporting varying degrees of success in controlling the drug release behavior, the sustained IOP lowering effect of anti-glaucoma agents from in situ gelling eye drops is limited (usually vanishes within 24 hours following administration). The medications should be employed by a route other than topical due to the static and dynamic barriers caused by the unique corneal anatomy and physiology (Ripal Gaudana et al. (2010), The AAPS Journal, 12(3):348-360). Intracameral administration is considered to be a more appropriate route that allows direct entry of bioactive substances into the anterior chamber and achieves higher drug levels at the site of action. By this way, multiple injections may be required to overcome the rapid elimination of free drugs through aqueous turnover and uveal blood flow. However, it has been documented that repeated intravitreal administrations over a period of time appear to increase risks of complications, including cataract, vitreous hemorrhage, and retinal detachment (Kohei Hironaka et al. (2009), Journal of Controlled Release, 136(3):247-253).

In ophthalmology, injectable in situ forming biomaterials have been mainly explored as intraocular lenses and vitreous substitutes (Hyder A. Aliyar et al. (2005), Biomacromolecules, 6(1):204-211). For delivery of therapeutics into the eye, the injectable gelling systems may offer a number of advantages not only to establish a minimally invasive surgery but also to prolong the drug residence time.

More recently, Turturro et al. reported that the PNIPAAm-based materials could be developed as injectable drug delivery platforms for the treatment of posterior segment diseases (Sanja B. Turturro et al. (2011), Biomaterials, 32:3620-3626). Their study demonstrates that the intravitreal injection of poly(ethylene glycol)diacrylate cross-linked PNIPAAm hydrogels causes a small transient effect on retinal function, but resolves rapidly within 7 days post operation. Although the lack of long-term changes in retinal cellular parameters has been suggested as indicative of acceptable biocompatibility of hydrogels, the PNIPAAm is not biodegradable in nature and may raise toxicity concerns related to its continued residence in the intraocular cavity. A previous investigation even showed that the extent of retinal destruction by 2 weeks after surgical replacement of rabbit vitreous with Pluronic F127 strongly depends on the concentration of thermo-responsive polymers (Frederick H. Davidoff et al. (1990), Retina, 10(4):297-300).

In Macromol. Biosci. (2006), 6(12):1026-1039, Jyh-Ping Chen et al. reported the synthesis of thermo-responsive comb-like polymers with chitosan as the backbone and pendant poly(N-isopropylacrylamide) (PNIPAM) groups by grafting PNIPAM-COOH with a single carboxy end group onto chitosan through amide bond linkages, in which PNIPAM-COOH was prepared by the free radical polymerization of NIPAM monomers and mercaptoacetic acid (MAA, acting as a chain transfer agent) with 2,2′-azoisobutyronitrile (AIBN) as an initiator. The synthesized thermo-responsive chitosan-graft-PNIPAM (CPN) copolymers were evaluated for their potential use as an injectable scaffold for the culture of articular chondrocytes and meniscus cells.

In Biomacromolecules (2010), 11(9):2261-2267, Scott D. Fitzpatrick et al. synthesized two thermoresponsive, bioactive cell scaffolds by decorating the backbone of type I bovine collagen with linear chains of poly(N-isopropylacrylamide) (PNIPAAm), in which NIPAAm monomers were polymerized using cysteamine hydrochloride as a chain transfer agent and N,N′-azobisisobutyronitrile (AIBN) as an initiator, and the resultant amine-terminated PNIPAAm was grafted onto the backbone of type I bovine collagen using 1-ethyl-(3-3-dimethylaminopropyl)carbodimiide hydrochloride (EDC) and N-hydroxysuccinimide (NHS) chemistry. The resultant thermoresponsive PNIPAAm-collagen copolymers were examined as cell-carrying biomaterial scaffolds aimed at improving outcomes in retinal-cell therapy for the treatment of retinal degenerative diseases.

By avoiding the need for surgical removal of foreign materials, bioerodible carriers can provide enhanced patient compliance and minimize undesirable tissue reactions following drug administration. Gelatin, a naturally occurring biopolymer derived from collagen, is a good candidate for ophthalmic applications (Jui-Yang Lai (2010), J. Mater. Sci.: Mater Med., 21(6):1899-1911). As early as 1993, Sreekant R. Nadkarni et al. proposed the use of absorbable gelatin device (i.e., Gelfoam sponge) for controlled ophthalmic delivery of pilocarpine (Sreekant R. Nadkarni et al. (1993), Pharm Res., 10(1):109-112). In Polym. Adv. Technol. (1998), 9(2):155-158, Hiroshi Yoshioka et al. reported a polymer conjugate composed of 43 wt % gelatin and 57 wt % poly(N-isopropylacrylamide) (PNIPAAm). To prepare said polymer conjugate, gelatin was treated with N-acryloylsuccinimide so as to introduce acryloyl groups into the gelatin. The resultant acrylated gelatin was then copolymerized with NIPAAm monomers in the presence of N,N,N′,N-tetramethylethylenediamine and ammonium persulfate. While said polymer conjugate was expected to be not only a safer biological glue but also an extracellular matrix for three-dimensional cell culture system which enables cells to be embedded and recovered by just changing temperature, only the dynamic viscoelastic properties thereof in an aqueous solution at the concentration of 5 wt % were examined.

In J. Biomater. Sci. Polymer Edn, 13(2):167-183 (2002), Nobuyuki Morikawa et al. reported the preparation of poly(N-isopropylacrylamide)-graft-copolymerized gelatin (PNIPAM-gelatin) by quasi-living photo-graft polymerization initiated from a photoinitiator multiply derivatized on gelatin molecules, in which gelatin was multiply derivatized with a photoiniferter (i.e., 4-(N,N-diethyldithiocarbamylmethyl)benzoic acid), and the resultant multiply dithiocarbamylated gelatin was subsequently copolymerized with NIPAAm monomers via quasi-living radical photopolymerization initiated by UV light, so that each of the PNIPAAm graft chains was bound to the gelatin molecule via a linking group —NH—(C═O)-Ph-CH₂—. In Shoji Ohya et al. (2005), J. Biomater. Sci. Polymer Edn., 16 (7):809-827, a series of PNIPAM-gelatins of this type were synthesized and examined their potential usefulness of as an in situ formable extracellular matrix (ECM).

Vandervoort et al. reported that topical application of bioadhesive gelatin nanoparticles loaded with pilocarpine could achieve a prolonged drug residence time at the eye surface (J. Vandervoort and A. Ludwig (2004), Eur. J. Pharm. Biopharm., 57(2):251-261). A study from Kuniko Hori et al. showed that the sustained release of epidermal growth factor from cationized gelatin hydrogels placed over the rabbit corneal epithelial defect significantly enhanced ocular surface wound healing (Kuniko Hori et al. (2007), Journal of Controlled Release, 118(2): 169-176).

There also exist numerous published patent documents disclosing hydrogel-forming sustained-release preparation (U.S. Pat. No. 6,436,441 B1), sustained release intraocular drug delivery system (US 20080107694 A1), biocompatible intraocular drug delivery systems (US20100311808 A1), injectable drug delivery formulation (US20110236454 A1), intracameral sustained release therapeutic agent implants (US20110182966 A1), etc. These patent documents are incorporated herein by reference in their entirety.

In this invention, the applicants attempted to provide a new biodegradable in situ forming DDS for intracameral administration of anti-glaucoma medications.

### SUMMARY OF THE INVENTION

Therefore, in a first aspect, this invention provides a hydrogel-forming polymer that forms in situ a biodegradable thermo-responsive hydrogel in an aqueous medium having a physiological temperature, the hydrogel-forming polymer comprising a reaction product of a biodegradable component having amino functional groups and a thermo-responsive component having a carboxylic acid end group, wherein:

the biodegradable component is a protein selected from the group consisting of natural gelatin, aminated gelatin, natural silk protein, aminated silk protein, or a combination thereof; and

the thermo-responsive component has a lower critical solution temperature lower than the physiological temperature and comprises a polymerization reaction product of a (meth)acrylamide monomer in the presence of a compound having a carboxyl group in the molecule thereof.

In a second aspect, this invention provides a process of making said hydrogel-forming polymer, the process comprising:

reacting a biodegradable component having amino functional groups with a thermo-responsive component having a carboxylic acid end group, wherein:
the biodegradable component is a protein selected from the group consisting of natural gelatin, aminated gelatin, natural silk protein, aminated silk protein, or a combination thereof; and
the thermoresponsive component has a lower critical solution temperature lower than the physiological temperature and comprises a reaction product generated from a polymerization reaction of a (meth)acrylamide monomer in the presence of a compound having a carboxyl group in the molecule thereof; and
recovering the hydrogel-forming polymer thus formed.

In a third aspect, this invention provides a sustained-release pharmaceutical composition comprising a therapeutic agent and the aforesaid hydrogel-forming polymer as a carrier material.

In a fourth aspect, this invention provides a method for treating an eye disorder, comprising administering to an eye of a mammal in need of such treatment the aforesaid sustained-release pharmaceutical composition.

### BRIEF DESCRIPTION OF THE DRAWINGS

The above and other objects, features and advantages of this invention will become apparent with reference to the following detailed description and the preferred embodiments taken in conjunction with the accompanying drawings, in which:

FIG. 1 shows the number-average molecular weight (Mn) of carboxylic end-capped poly(N-isopropylacrylamide)(abbreviated as PN) as a function of MAA/NIPAAm molar ratio, in which MAA and NIPAAm refer to mercaptoacetic acid and N-isopropylacrylamide, respectively, and three different samples of PN were synthesized according to the procedures set forth in Section A of the Experimental Procedures of the Synthesis Examples, described infra, using a MAA/NIPAAm molar ratio of 0.1, 0.2 and 0.4, respectively; each error bar represents standard deviation calculated from triplicates; and an asterisk (*) above an error bar indicates statistically significant difference (P<0.05) as compared to the PN sample prepared at a MAA/NIPAAm molar ratio of 0.1 (i.e., the product obtained in Section A of the Experimental Procedures of the Synthesis Examples, described infra);

FIG. 2 shows the ¹H NMR spectra of gelatin and A-gelatin samples in DMSO, in which A-gelatin refers to an aminated gelatin product as obtained in Section B of the Experimental Procedures of the Synthesis Examples, described infra;

FIG. 3 shows the Fourier transform infrared spectroscopy (FTIR) spectra of gelatin, A-gelatin, NIPAAm, PN and GN samples, in which NIPAAm refers to N-isopropylacrylamide; A-gelatin refers to the product obtained in Section B of the Experimental Procedures of the Synthesis Examples, described infra; PN refers to the product obtained in Section A of the Experimental Procedures of the Synthesis Examples, described infra; and GN refers to a coupling (grafting) reaction product of A-gelatin and PN as obtained in Section C of the Experimental Procedures of the Synthesis Examples, described infra;

FIG. 4 shows the gross morphological changes of A-gelatin, PN and GN samples prepared in deionized water (upper panels: 2.5% w/v; lower panels: 10% w/v) when heating from 25° C. to 34° C., in which A-gelatin, PN and GN are the same material as those described in FIG. 3;

FIG. 5 shows the DSC thermograms of PN and GN samples as prepared in deionized water or BSS (10% w/v), in which PN and GN are the same material as those described in FIG. 3; and each lower critical solution temperature (LCST) data point represents the averaged results of five independent experiments;

FIG. 6 shows the time course absorbance changes for the PN and GN samples prepared in H₂O or BSS (10% w/v) in response to temperature increase from 25° C. to 34° C., in which PN and GN are the same material as those described in FIG. 3; and each gel formation time (GFT) data point represents the averaged results of five independent experiments;

FIG. 7 shows the time course of the weight remaining of a hydrogel sample formed from a test material (PN or GN) after incubation at 34° C. in balanced salt solution (BSS, pH 7.4) containing matrix metalloproteinase-2 (MMP-2), in which PN and GN are the same material as those described in FIG. 3; incubation time point: hour (h) and day (d); each error bar represents standard deviation calculated from triplicates; and an asterisk (*) indicates statistically significant differences (P<0.05) as compared to the PN sample at the same designated incubation time point;

FIG. 8 shows the phase-contrast micrographs of BCE C/D-1b and HLE-B3 cells after a 2-day exposure to an in situ formed hydrogel from a test material (PN or GN), in which PN and GN are the same material as those described in FIG. 3; cells cultured with culture medium only served as a control; and scale bar=100 μm;

FIG. 9 shows the fluorescence images of BCE C/D-1b and HLE-B3 cell cultures after a 2-day exposure to an hydrogel formed in situ from a test material (PN or GN), in which PN and GN are the same material as those described in FIG. 3; the cultured cells were examined by a cell viability assay using the Live/Dead Viability/Cytotoxicity Kit (Molecular Probes, Eugene, Oreg., USA), so that live cells showed green fluorescence and dead cells showed red fluorescence; cells cultured in culture medium only served as a control; and scale bar=100 μm;

FIG. 10 shows the cell proliferation results of BCE C/D-1b and HLE-B3 cells after a 2-day exposure to an hydrogel formed in situ from a test material (PN or GN), as examined by the cell proliferation MTS assay, in which PN and GN are the same material as those described in FIG. 3; the results were expressed as relative MTS activity as compared to the control group (i.e., cells cultured in culture medium only); each error bar represents standard deviation calculated from quadruplicates; and an asterisk (*) indicates statistically significant differences (P<0.05);

FIG. 11 shows the gene expression of IL-6 in BCE C/D-1b and HLE-B3 cells after a 2-day exposure to an hydrogel formed in situ from a test material (PN or GN), as determined by quantitative real-time reverse transcription polymerase chain reaction (RT-PCR), in which PN and GN are the same material as those described in FIG. 3; the gene expression of IL-6 was normalized with that of glyceraldehyde 3-phosphate dehydrogenase (GAPDH); the results were expressed as relative IL-6 mRNA level (%) as compared to the control group (i.e., cells cultured in culture medium only); and each error bar represents standard deviation calculated from triplicates;

FIG. 12 is a bar diagram showing the time course of the release of pilocarpine from a pilocarpine-incorporated hydrogel sample prepared from a test material (PN or GN), in which PN and GN are the same material as those described in FIG. 3; the pilocarpine-incorporated hydrogel sample was incubated at 34° C. in a release buffer (BSS containing MMP-2), which was subsequently collected and analyzed by HPLC at a designated incubation time point (h, hour; d, day); each error bar represents standard deviation calculated from quintuplicates; and an asterisk (*) indicates statistically significant differences (P<0.05) as compared to the pilocarpine-incorporated PN hydrogel sample at the same designated incubation time point;

FIG. 13 shows the cumulative release profile of pilocarpine as a function of time from a pilocarpine-incorporated hydrogel sample prepared from a test material (PN or GN), in which PN and GN are the same material as those described in FIG. 3; the pilocarpine-incorporated hydrogel sample was incubated at 34° C. in a release buffer (BSS containing MMP-2), which was subsequently collected and analyzed by HPLC at a designated incubation time point (h, hour; d, day); the experiment was performed in quintuplicate, and the cumulative release profile was plotted based on the calculated mean values at the indicated incubation time point;

FIG. 14 shows the representative slit-lamp biomicroscopic images of rabbit eyes, in which left panel, the rabbit eye before operation; middle panel, the rabbit eye after glaucoma induction; and right panel, a glaucomatous rabbit eye receiving intracameral administration of a GN-based drug delivery system (DDS) containing pilocarpine using a 30-gauge needle; and GN is the same material as that described in FIG. 3;

FIG. 15 shows the representative time-course slit-lamp biomicroscopic images of rabbit eyes in four test groups (Eye Drop, Free-Drug, PN-Drug and GN-Drug) at different time points (4 hours, 3 days and 2 weeks) after pilocarpine administration; in which Eye Drop group, topical administration of a test solution A containing 2% w/v pilocarpine nitrate dissolved in deionized water; Free-Drug group, intracameral administration of the test solution A; PN-Drug group, intracameral administration of a test solution B containing 2% w/v pilocarpine nitrate and 10% w/v PN dissolved in deionized water; and GN-Drug group, intracameral administration of a test solution C containing 2% w/v pilocarpine nitrate and 10% w/v GN dissolved in BSS deionized water; and scale bars=5 mm;

FIG. 16 shows typical specular microscopic images of rabbit corneal endothelium photographed at time points before (panel A) and after (panel B) glaucoma induction, and photographed after 2 weeks of pilocarpine administration for four test groups, in which Eye Drop group (panel D), Free-Drug group (panel E), PN-Drug group (panel F) and GN-Drug group (panel G) are the same as those described in FIG. 15; and glaucomatous rabbits receiving no drug served as a control group (panel C);

FIG. 17 shows the preoperative and postoperative specular microscopy measurements of corneal endothelial cell density for rabbits in different test groups, in which Pre, the rabbit eye before operation; GL, the rabbit eye after glaucoma induction; Ctrl, glaucomatous eye receiving no drug; the Eye Drop, Free-Drug, PN-Drug and GN-Drug groups are the same as those described in FIG. 15; each error bar represents standard deviation calculated from sextuplicates; and an asterisk (*) indicates statistically significant differences (P<0.05) as compared to the GL group;

FIG. 18 shows the measurements of the intraocular pressure (IOP) after pilocarpine administration for different test groups, in which the Eye Drop, Free-Drug, PN-Drug and GN-Drug groups are the same as those described in FIG. 15; and glaucomatous rabbits receiving no drug served as a control group (Ctrl); follow-up time points: Pre, before operation; h, hour; and d, day; each error bar represents standard deviation calculated from sextuplicates; and an asterisk (*) indicates statistically significant differences (P<0.05) as compared to the Ctrl group; and

FIG. 19 shows the measurements of pupil diameter after pilocarpine administration for different test groups, in which the Eye Drop, Free-Drug, PN-Drug and GN-Drug groups are the same as those described in FIG. 15; and glaucomatous rabbits receiving no drug served as a control group (Ctrl); follow-up time points: Pre, before operation; h, hour; and d, day; each error bar represents standard deviation calculated from sextuplicates; and an asterisk (*) indicates statistically significant differences (P<0.05) as compared to the Ctrl group.

### DETAILED DESCRIPTION OF THE INVENTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless defined otherwise, all technical and scientific terms used herein have the meaning commonly understood by a person skilled in the art to which this invention belongs. One skilled in the art will recognize many methods and materials similar or equivalent to those described herein, which could be used in the practice of this invention. Indeed, this invention is in no way limited to the methods and materials described.

As used herein, the term “hydrogel-forming polymer” refers to any polymer which upon contact with an aqueous medium is capable of forming a hydrogel, i.e., a swollen, water-containing network of polymer chains that are water-insoluble. In particular, the hydrogel-forming polymer comprises hydrophilic groups that are capable of forming strong hydrogen bonds with water, such as —C—O—C—, —C—O—H, —C—N—C— or —C═O.

As used herein, the term “aqueous medium” refers to a liquid mass comprising substantial proportion of water that can also contain either dissolved or suspended organic or inorganic matter. The term “aqueous medium” is intended to encompass both fresh water and salt water, including any fluid having water as the continuous phase, including oil-in-water emulsions, etc. The term “aqueous medium” comprises water, buffers and solutions in general wherein water is used as solvent. The term “aqueous medium” includes media containing water or another hydrophilic and water-miscible substance such as, e.g., glycerol. The term “aqueous medium” also includes any pharmaceutically acceptable dissolution medium, and any body fluid which bathes or surrounds living cells or tissues. Body fluids include aqueous humour and vitreous humour, bile, blood serum, breast milk, cerebrospinal fluid, cerumen (earwax), endolymph and perilymph, female ejaculate, gastric juice, mucus (including nasal drainage and phlegm), peritoneal fluid, pleural fluid, saliva, sebum (skin oil), semen, sweat, tears, vaginal secretion, vomit, urine, etc.

As used herein, the term “physiological temperature,” except as otherwise indicated, refers to the range in body temperatures found in mammals, including humans, cattle, swine, horses, goats, sheep, dogs, cats, rabbits, etc. The physiological temperature of mammals is generally within the range of from about 32° C. to about 42° C. The physiological temperatures for some exemplary mammals are as follows: humans, 37° C.; dogs, 39° C.; cats, 38° C.; pigs, 37° C.; horses, 37° C.; cattle, 39° C.; and goats, 39° C. The term “physiological temperature” is taken herein to refer most specifically to temperatures in the range 30-42° C., and preferably 34-40° C.

As used herein, the term “biodegradable polymer” means a polymer or polymers which degrade in vivo, and wherein erosion of the polymer or polymers over time occurs concurrent with or subsequent to release of therapeutic agent(s). The terms “biodegradable” and “bioerodible” are used interchangeably herein. A biodegradable polymer may be a homopolymer, a copolymer, or a polymer comprising more than two different polymeric units. The polymer can be a gel or hydrogel type polymer.

As used herein, the term “lower critical solution temperature (LCST)” refers to a temperature of a polymer, below which the polymer and solvent are completely miscible and form a single phase. For example, “the LCST of a polymer solution” means that the polymer is uniformly dispersed in a solution at a specified temperature (i.e., LCST) or lower. The polymer aggregates and forms a second phase when the solution temperature is increased beyond the specified temperature (i.e., LCST).

As used herein, the term “thermo-responsive polymer (TRP)” refers to a polymer that undergoes a physical change when external thermal stimuli are presented. The polymer may change some of its properties at a particular temperature. One such property may be the solubility of a polymer in a particular solvent. For example, a solution of a TRP may be prepared by solubilizing a TRP in a solvent below the LCST of the TRP, where the solution phase of the TRP is stable. When the temperature of the TRP solution is increased above the LCST, the solution phase may become unstable and a second phase forms. Therefore, the TRP may become less soluble (more hydrophobic) in the solvent, such as, in water, at a temperature higher than LCST. Changing the pH and the ionic strength may affect the LCST of a TRP.

A non-limiting example of a TRP is poly(N-isopropylacrylamide) (PNIPAAm). Under standard conditions of neutral pH and in the absence of ionic species, PNIPAAm undergoes a phase transition from soluble to insoluble form at a temperature of ˜32° C. The application of TRPs, especially PNIPAAm, has been explored in cell culture and tissue engineering because of its LCST (˜32° C.), which is close to physiological temperature of 37° C. At this temperature, the polymer is hydrophobic, which helps cells to adhere on its surface. When the temperature is lowered to below the LCST, the polymer becomes hydrophilic and swollen, which triggers detachment of the cells.

According to this invention, the term “carboxylic end-capped poly(N-isopropylacrylamide)” is used interchangeable with the term “carboxylated poly(N-isopropylacrylamide)” and is abbreviated as “PNIPAAm-COOH” or “PN” hereinafter. The term “carboxylic end-capped poly(N-isopropylacrylamide)” refers to a polymer of N-isopropylacrylamide having a carboxylic acid end group that is introduced into the polymer's molecule by a compound having a carboxyl group in the molecule thereof, in particular a chain transfer agent such as mercaptoacetic acid, during polymerization of N-isopropylacrylamide. This definition for the term “carboxylic end-capped poly(N-isopropylacrylamide)” also applies to other carboxylic end-capped polymers encompassed by the concept of this invention.

As used herein, the term “mammal” refers to an organism of the order Mammalia, including humans, primates, and non-primates such as dogs, cats, horses, cattle, rabbits, mice, rats, guinea pigs, hamsters, marsupials and the like.

As used herein, the term “therapeutic agent” is defined as a substance capable of administration to an animal, preferably a human, which modulates the animal's physiology. More preferably, the term “therapeutic agent,” as used herein, is defined as any substance intended for use in the treatment or prevention of disease in an animal, preferably in a human. Therapeutic agent includes synthetic and naturally occurring bioaffecting substances, as well as recognized pharmaceuticals, such as those listed in “The Physicians Desk Reference,” 61st edition (2007), “Goodman and Gilman's The Pharmacological Basis of Therapeutics” 10th Edition (2001), and “The United States Pharmacopeia, The National Formulary”, USP XXX NF XXV (2007), the compounds of these references being herein incorporated by reference. The term “therapeutic agent” also includes compounds that have the indicated properties that are not yet discovered. The term “therapeutic agent” includes pro-active, activated and metabolized forms of therapeutic agents.

In earlier studies, the applicants demonstrated the potential of multifunctional gelatin carriers for intraocular delivery of cell/tissue sheets (Jui-Yang Lai et al. (2006), Biomacromolecules, 7(6):1836-1844; Jui-Yang Lai et al. (2009), Biomacromolecules, 10(2):310-319). In light of these promising results, the applicants turned to investigate the applicability of PNIPAAm-grafted gelatin (GN copolymer) as biodegradable in situ forming DDSs for intracameral administration of anti-glaucoma medications. The applicants hypothesized that the biodegradable features and the temperature-sensitive features owned by the GN copolymer will permit efficient intraocular delivery of therapeutics, thereby giving an increase in performance over either eye drop instillation or free drug injection.

To verify this hypothesis, the applicants synthesized an aminated gelatin and grafted it with carboxylic end-capped PNIPAAm (PN) via a carbodiimide-mediated coupling reaction. The GN copolymer thus obtained was admixed with pilocarpine in an aqueous solution at 25° C., and the resultant liquid was subsequently injected into the anterior chamber of a glaucomatous rabbit's eye having a temperature of 34° C., causing a rapid in situ formation of a drug-incorporated hydrogel inside the anterior chamber of the rabbit's eye by virtue of temperature triggered sol-gel phase transition. Gradual biodegradation of the drug-incorporated hydrogel was observed, allowing a sustained release of pilocarpine for an extended period of time over two weeks.

Accordingly, this invention provides a hydrogel-forming polymer that forms in situ a biodegradable thermo-responsive hydrogel in an aqueous medium having a physiological temperature. The hydrogel-forming polymer of this invention comprises a reaction product of a biodegradable component having amino functional groups and a thermo-responsive component having a carboxylic acid end group. The hydrogel-forming polymer of this invention can be made by a process comprising:

reacting a biodegradable component having amino functional groups with a thermo-responsive component having a carboxylic acid end group, wherein:
the biodegradable component is a protein selected from the group consisting of natural gelatin, aminated gelatin, natural silk protein, aminated silk protein, or combinations thereof; and
the thermo-responsive component has a lower critical solution temperature lower than the physiological temperature and comprises a reaction product generated from a polymerization reaction of a (meth)acrylamide monomer in the presence of a compound having a carboxyl group in the molecule thereof; and

recovering the hydrogel-forming polymer thus formed.

The term “reaction product” as used herein refers to one or more compounds formed by the reaction of one, two or more reactants. For example, in this invention, the term “reaction product” may include more than one chemical compound formed from at least one type of (meth)acrylamide monomer and at least one type of the compound having a carboxyl group in the molecule thereof.

According to this invention, the step of reacting the biodegradable component with the thermo-responsive component is conducted in the presence of a coupling reagent. According to this invention, the coupling reagent may be 1-ethyl-3-(3-dimethylaminopropyl)carbodiimide (EDC), N,N′-dicyclohexylcarbodiimide (DCC), N,N′-diisopropylcarbodiimide (DIC), or combinations thereof. In a preferred embodiment of this invention, the coupling reagent is EDC.

According to this invention, the step of reacting the biodegradable component with the thermo-responsive component is conducted in the presence of an activating reagent for carboxylic acids. According to this invention, the activating reagent for carboxylic acids may be N-hydroxysuccinimide (NHS), N-hydroxysulfosuccinimide (Sulfo-NHS), 1-hydroxybenzotriazole (HOBt), 1-hydroxy-7-azabenzotriazole (HOAt), pentafluorophenol), or combinations thereof. In a preferred embodiment of this invention, the activating reagent for carboxylic acids is NHS.

In a preferred embodiment of this invention, the step of reacting the biodegradable component with the thermo-responsive component is conducted in the presence of both EDC and NHS.

The molecular weight of the biodegradable component may influence the biodegradation rate thereof, namely the smaller its molecular weight was, the faster the biodegradation thereof would be. Therefore, in order to provide the sustained release effect, the biodegradable component of this invention has a weight average molecular weight (Mw) in a range from 10,000 to 1,000,000, preferably from 10,000 to 300,000, more preferably from 50,000 to 200,000, and most preferably from 50,000 to 150,000. In a preferred embodiment of this invention, the biodegradable component has a weight average molecular weight of ˜100,000.

While natural gelatin and natural silk protein each already have amino functional groups in the molecule thereof, these two proteins may be aminated so as to increase the number of amino functional groups thereof, thereby increasing the amount/number of the thermo-responsive component coupled thereto. In this aspect, amination of natural gelatin and natural silk protein may be effected by using a dihydrazide compound, a diamine compound, or combinations thereof, to convert the carboxylic functional groups thereof into amino functional groups, as schematically shown below.

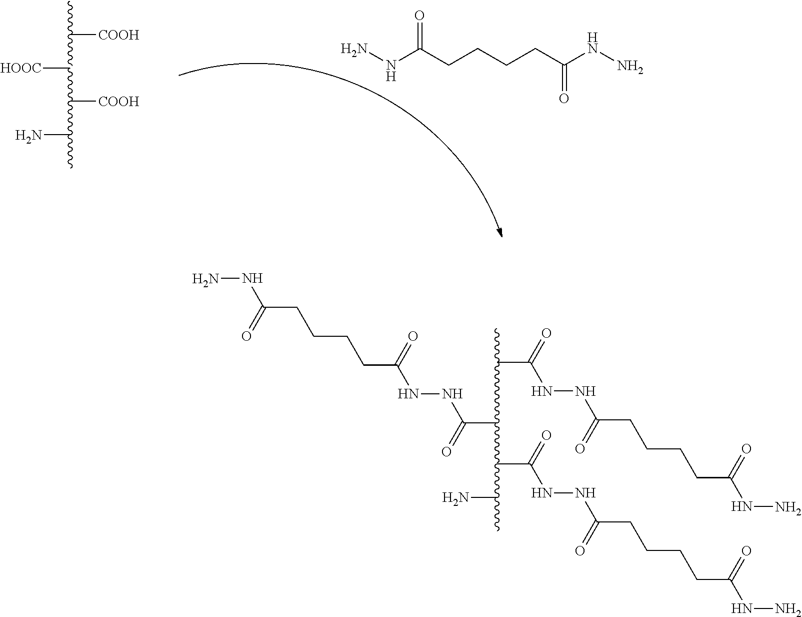

According to this invention, the dihydrazide compound may be adipic acid dihydrazide, succinic acid dihydrazide, sebacic acid dihydrazide, valine dihydrazide, isophthalic dihydrazide, carbodihydrazide, icosanedioic acid dihydrazide, or combinations thereof.

According to this invention, the diamine compound may be ethylenediamine, propane-1,3-diamine, butane-1,4-diamine, pentane-1,5-diamine, hexane-1,6-diamine, or combinations thereof.

In a preferred embodiment of this invention, the biodegradable component is gelatin or aminated gelatin. In a more preferred embodiment of this invention, the biodegradable component is aminated gelatin. In a more preferred embodiment of this invention, the biodegradable component is aminated gelatin formed by aminating natural gelatin with adipic acid dihydrazide.

Preferably, the number of amino functional groups in the biodegradable component is in a range from 10 to 200 per mole of the biodegradable component. More preferably, the number of amino functional groups in the biodegradable component is in a range from 30 to 200 per mole of the biodegradable component.

The (meth)acrylamide monomer suitable for use in generating the thermo-responsive component of this invention includes N-isopropylacrylamide, N,N-diethylacrylamide, N-n-propylacrylamide, acrylamide, N-isopropylmethacrylamide, methacrylamide, N-methylacrylamide, N-methylmethacrylamide, N-ethylacrylamide, N-ethylmethacrylamide, N-n-propylmethacrylamide, N,N-dimethylmethacrylamide, N,N-dimethylacrylamide, N-n-butylacrylamide, or combinations thereof.

In a preferred embodiment of this invention, the (meth)acrylamide monomer is N-isopropylacrylamide. In another preferred embodiment of this invention, the (meth)acrylamide monomer is N,N-diethylacrylamide. In a further preferred embodiment of this invention, both N-isopropylacrylamide and N,N-diethylacrylamide are used to generated the thermo-responsive component of this invention. Accordingly, the reaction product comprised in the thermo-responsive component of this invention may be a carboxylic end-capped polymer of N-isopropylacrylamide, a carboxylic end-capped polymer of N,N-diethylacrylamide, a carboxylic end-capped copolymer of N-isopropylacrylamide and N,N-diethylacrylamide, or combinations thereof.

According to this invention, the compound having a carboxyl group in the molecule thereof is a chain transfer agent selected from the group consisting of mercaptoacetic acid (MAA), 2-mercaptopropionic acid (2-MPA), 3-mercaptopropionic acid (3-MPA), 4-mercaptobutyric acid, 5-mercaptopentanoic acid, 6-mercaptohexanoic acid, p-mercaptobenzoic acid, 3,3′-dithiodipropionic acid (DTDPA), mercaptosuccinic acid (MSA), 5,5′-dithiobis(2-nitrobenzoic acid) (DNBA), 11-mercaptoundecanoic acid (MUA), or combinations thereof. In a preferred embodiment of this invention, the chain transfer agent is MAA.

According to this invention, the polymerization reaction of the (meth)acrylamide monomer may be initiated by irradiation with UV or radioactive rays, or by a polymerization initiator.

In a preferred embodiment of this invention, the polymerization reaction of the (meth)acrylamide monomer is initiated by irradiation with UV or radioactive rays using 1-azobis(4-cyanovaleric acid) as the compound having a carboxyl group in the molecule thereof.

In another preferred embodiment of this invention, the polymerization reaction of the (meth)acrylamide monomer is initiated by a radical generation type polymerization initiator such as an azo compound or a peroxide. The azo compound suitable for use in this invention includes 2,2′-azobisisobutyronitrile (AIBN), 2,2′-azobis(cyclohexanecarbonitrile), 2,2-azobis(2-methylbutyronitrile), 2,2′-azobis(2,4-dimethylvaleronitrile), 1-azobis(1-cyclohexanecarbonitrile), 1-azobis(4-cyanovaleric acid), or combinations thereof. In a preferred embodiment of this invention, the azo compound is AIBN.

In a preferred embodiment of this invention, the (meth)acrylamide monomer is N-isopropylacrylamide, the compound having a carboxyl group in the molecule thereof is mercaptoacetic acid, and the reaction product comprised in the thermo-responsive component is a carboxylic end-capped poly(N-isopropylacrylamide) generated from the polymerization reaction of N-isopropylacrylamide in the presence of mercaptoacetic acid.

The gelation behavior of the thermo-responsive component of this invention will be influenced by the molecular weight thereof, namely the higher its molecular weight is, the thermo-responsive component forms a hydrogel more easily, whereas the lower its molecular weight is, the thermo-responsive component forms precipitates more easily. Therefore, the thermo-responsive component of this invention preferably has a number average molecular weight in a range from 1,000 to 10,000, more preferably from 3,000 to 10,000, and most preferably from 5,000 to 10,000. In a preferred embodiment of this invention, the thermo-responsive component has a number average molecular weight of around 7,400.

The molecular weight of the thermo-responsive component of this invention can be controlled by adjusting the used molar ratio of the compound having a carboxyl group in the molecule thereof to the (meth)acrylamide monomer, preferably in a range from 0.01 to 0.5, more preferably in a range from 0.05 to 0.4, and most preferably in a range from 0.05 to 0.2. In a preferred embodiment of this invention, the used molar ratio of the compound having a carboxyl group in the molecule thereof to the (meth)acrylamide monomer is 0.1.

The thermo-responsive component of this invention has a lower critical solution temperature (LCST) in a range preferably from 20 to 40° C., and more preferably from 25 to 35° C. In a preferred embodiment of this invention, the thermo-responsive component has a LCST of ˜32° C.

According to this invention, the biodegradable component and the thermo-responsive component may react with each other at a temperature ranging from 20 to 25° C., eliciting a condensation reaction between the carboxylic acid end group of the thermo-responsive component and one of the amino functional groups of the biodegradable component, in view of existing literature (see, for example, Jyh-Ping Chen et al. (2006), Macromol. Biosci., 6:1026-1039; and Scott D. Fitzpatrick et al. (2010), Biomacromolecules, 11(9):2261-2267). Thus, in the recovered hydrogel-forming polymer, the thermo-responsive component is coupled to the biodegradable component via a covalent bond formed between the carboxylic acid end group of the thermo-responsive component and one of the amino functional groups of the biodegradable component.

In order to confer the hydrogel-forming polymer of this invention with rapid in situ gelling ability that imparts better delivery capabilities, the process of this invention is performed at a NH₂/COOH molar ratio preferably not greater than 1, more preferably not greater than 0.8, more preferably not greater than 0.5, and most preferably not greater than 0.36. In a preferred embodiment of this invention, the biodegradable component is reacted with the thermo-responsive component at a NH₂/COOH molar ratio of 0.36.

Due to the LCST of the thermo-responsive component, the hydrogel-forming polymer of this invention is likewise thermo-responsive and displays temperature triggered sol-gel phase transition in aqueous media including water, buffered solutions such as balanced salt solution (BSS), and body fluids such as aqueous humour.

According to the in vitro and in vivo pharmaceutical experiments performed, the hydrogel-forming polymer of this invention was shown to have potential to act as a carrier material in sustained-release pharmaceutical compositions. Therefore, this invention further provides a sustained-release pharmaceutical composition comprising the hydrogel-forming polymer described above as a carrier material, and a therapeutic agent.

Depending on the required drug release rate and drug encapsulating rate, a person with ordinary skill in the art may synthesize the hydrogel-forming polymer of this invention that meets these requirements by adjusting one or more of the following: the type of protein used as the biodegradable component, the molecular weight of the biodegradable component, the type of the (meth)acrylamide monomer used to synthesize the thermo-responsive component, the molecular weight of the thermo-responsive component, and the NH₂/COOH molar ratio.

For the sake of example only, the therapeutic agent includes pharmaceutically active compounds, hormones, growth factors, enzymes, DNA, plasmid DNA, RNA, siRNA, viruses, proteins, lipids, pro-inflammatory molecules, antibodies, antibiotics, anti-inflammatory agents, anti-sense nucleotides, transforming nucleic acids, living cells, or combinations thereof. Any of the therapeutic agents may be combined to the extent such combination is biologically compatible.

In a preferred embodiment of this invention, the therapeutic agent is selected from the group consisting of anesthetics, analgesics, dopaminergic antagonists, anticancer agents, anti-proliferative agents, angiogenesis inhibitors, anti-infective agents, anti-inflammatory agents, antiviral agents, antibiotics, immunomodulatory agents, hormones, or combinations thereof.

The sustained-release pharmaceutical composition of this invention may be delivered to a target site in a mammal in need of treatment via a parenteral route, including, but not limited to: anterior chamber injection, intra-retinal injection, subretinal injection, intravitreal injection, suprachoroidal injection, subcutaneous injection, intraepidermal injection, intramuscular injection, intrapentoneal injection, intraperitoneal injection, intrapleural injection, intrasynovial injection, intraarticular injection, intrasternal injection, intrathecal injection, intralesional injection, and intracranial injection.

For parenteral administration, the sustained-release pharmaceutical composition of this invention is manufactured as injectables, i.e., solutions, suspensions, and dry powders for reconstitution.

A solution for injection is a mixture of two or more components that form a single phase that is homogeneous down to the molecular level. “Water for injection” is the most widely used solvent for parenteral formulations. However, a nonaqueous solvent or a mixed aqueous/nonaqueous solvent system may be necessary to stabilize drugs that are readily hydrolyzed by water or to improve solubility. A range of excipients may be included in parenteral solutions, including antioxidants, antimicrobial agents, buffers, chelating agents, inert gases, and substances for adjusting tonicity. Antioxidants maintain product stability by being preferentially oxidized over the shelf life of the product.

Antimicrobial preservatives inhibit the growth of any microbes that are accidentally introduced while doses are being withdrawn from multiple-dose bottles and act as adjuncts in aseptic processing of products. Buffers are necessary to maintain both solubility of the active ingredient and stability of the product. Chelating agents are added to complex and thereby inactivate metals, including copper, iron, and zinc, which generally catalyze oxidative degradation of drugs. Inert gases are used to displace the air in solutions and enhance product integrity of oxygen-sensitive drugs. Isotonicity of the formulation is achieved by including a tonicity-adjusting agent. Failing to adjust the tonicity of the solution can result in the hemolysis or crenation of erythrocytes when hypotonic or hypertonic solutions, respectively, are given intravenously (IV) in quantities>100 mL. Injectable formulations must be sterile and free of pyrogens. Pyrogenic substances are primarily lipid polysaccharides derived from microorganisms, with those produced by gram-negative bacilli generally being most potent.

A suspension for injection consists of insoluble solid particles dispersed in a liquid medium, with the solid particles accounting for 0.5-30% of the suspension. The vehicle may be aqueous, oil, or both. Caking of injectable suspensions is minimized through the production of flocculated systems, comprising clusters of particles (flocs) held together in a loose open structure. Excipients in injectable suspensions include antimicrobial preservatives, surfactants, dispersing or suspending agents, and buffers. Surfactants wet the suspended powders and provide acceptable syringeability while suspending agents modify the viscosity of the formulation. The ease of injection and the availability of the drug in depot therapy are affected by the viscosity of the suspension and the particle size of the suspended drug. These systems afford enhanced stability to active ingredients that are prone to hydrolysis in aqueous solutions. Injectable suspensions are commonly used. Compared with that of injectable solutions, the rate of drug absorption of injectable suspensions is prolonged because additional time is required for disintegration and dissolution of the suspended drug particles. The slower release of drug from an oily suspension compared with that of an aqueous suspension is attributed to the additional time taken by drug particles suspended in an oil depot to reach the oil/water boundary and become wetted before dissolving in tissue fluids.

A dry powder for parenteral administration is reconstituted as a solution or as a suspension immediately prior to injection. The principal advantage of this dosage form is that it overcomes the problem of instability in solution.

In a preferred embodiment of this invention, the sustained-release pharmaceutical composition is manufactured as an injectable liquid, in particular a sterile aqueous solution or suspension for parenteral injection. In another preferred embodiment of this invention, the sustained-release pharmaceutical composition is manufactured as a dry powder for parenteral injection.

In a preferred embodiment of this invention, the hydrogel-forming polymer and the therapeutic agent contained in the sustained-release pharmaceutical composition are packaged separately as a kit and are admixed together with a pharmaceutically acceptable carrier prior to administering to a mammal in need of treatment.

When the sustained-release pharmaceutical composition of this invention is manufactured as an injectable, the hydrogel-forming polymer and the therapeutic agent are admixed with one or more pharmaceutically acceptable carriers, including, but not limited to: solvent, such as water; buffer, such as ophthalmic balanced salt solution, phosphate buffered saline (PBS), Ringer's solution, Hank's solution; emulsifier; suspending agent; decomposer; pH adjusting agent; stabilizing agent; chelating agent; preservative; diluent; absorption delaying agent; liposome; and the like. Selection and use of these pharmaceutically acceptable carriers will be readily apparent to those practitioners of ordinary skill in the art.

According to this invention, in the sustained-release pharmaceutical composition, the weight ratio of the hydrogel-forming polymer to the therapeutic agent is in a range preferably from 20:1 to 1:1, and more preferably from 10:1 to 5:2. In a preferred embodiment of this invention, the weight ratio of the hydrogel-forming polymer to the therapeutic agent is 5:1.

In a preferred embodiment of this invention, sustained-release pharmaceutical composition is for use in the treatment or prevention of an ophthalmic disorder or condition, and the therapeutic agent contained therein is an ophthalmic drug.

Therefore, this invention further provides a method for treating or preventing an ophthalmic disorder, comprising intraocularly administering into an eye of a mammal in need of such treatment a sustained-release pharmaceutical composition that comprises an ophthalmic drug and a hydrogel-forming polymer as described above.

As used herein, the term “ophthalmic disorder” and/or “ophthalmic condition” refers to ophthalmic diseases, conditions and/or disorders, including, without limitation, those associated with the anterior chamber of the eye (i.e., hyphema, synechia); the choroid (i.e., choroidal detachment, choroidal melanoma, multifocal choroidopathy syndromes); the conjunctiva (i.e., conjunctivitis, cicatricial pemphigoid, filtering Bleb complications, conjunctival melanoma, Pharyngoconjunctival Fever, pterygium, conjunctival squamous cell carcinoma); connective tissue disorders (i.e., ankylosing spondylitis, pseudoxanthoma elasticum, corneal abrasion or edema, limbal dermoid, crystalline dystrophy keratits, keratoconjunctivitis, keratoconus, keratopathy, megalocornea, corneal ulcer); dermatologic disorders (i.e., ecrodermatitis enteropathica, atopic dermatitis, ocular rosacea, psoriasis, Stevens-Johnson syndrome); endocrine disorders (i.e., pituitary apoplexy); extraocular disorders (i.e., Abducens Nerve Palsy, Brown syndrome, Duane syndrome, esotropia, exotropia, oculomotor nerve palsy); genetic disorders (i.e., albinism, Down syndrome, Peters Anomaly); the globe (i.e., anophthalmos, endophthalmitis); hematologic and cardiovascular disorders (i.e., Giant Cell Arteritis, hypertension, leukemias, ocular ischemic syndrome, sickle cell disease); infectious diseases (i.e., actinomycosis, botulism, HIV, diphtheria, Escherichia coli, Tuberculosis, ocular manifestations of syphilis); intraocular pressure (i.e., glaucoma, ocular hypotony, Posner-Schlossman syndrome), the iris and ciliary body (i.e., aniridia, iris prolaps, juvenile xanthogranuloma, ciliary body melanoma, iris melanoma, uveitis); the lacrimal system (i.e., alacrima, Dry Eye syndrome, lacrimal gland tumors); the lens (i.e., cataract, ectopia lentis, intraocular lens decentration or dislocation); the lid (i.e., blepharitis, dermatochalasis, distichiasis, ectropion, eyelid coloboma, Floppy Eye syndrome, trichiasis, xanthelasma); metabolic disorders (i.e., gout, hyperlipoproteinemia, oculocerebrorenal syndrome); neurologic disorders (i.e., Bell Palsy, diplopia, multiple sclerosis); general ophthalmologic (i.e., red eye, cataracts, macular degeneration, red eye, macular degeneration); the optic nerve (i.e., miningioma, optic neuritis, optic neuropathy, papilledema); the orbit (i.e., orbital cellulits, orbital dermoid, orbital tumors); phakomatoses (i.e., ataxia-telangiectasia, neurofibromatosis-1); presbyopia; the pupil (i.e., anisocoria, Horner syndrome); refractive disorders (i.e., astigmatism, hyperopia, myopia); the retina (i.e., Coats disease, Eales disease, macular edema, retinitis, retinopathy); and the sclera (i.e., episcleritis, scleritis).

According to this invention, the ophthalmic drug includes, but is not limited to: anti-glaucoma agents, anti-cataract agents, dopaminergic antagonists, beta adrenergic blockers, angiogenesis inhibitors, anti-infective agents, antibiotics, antibacterials, antivirals, anti-proliferative agents, anti-allergenics, anti-inflammatory agents, hormonal agents, growth factors, carbonic anhydrase inhibitors, decongestants, miotics, anticholinesterase, mydriatics, sympathomimetics, mucus secretogogue, immunomodulatory agents, mast cell stabilizers, etc.

Examples of ophthalmic drugs include antibiotics such as tetracycline, chlortetracycline, bacitracin, neomycin, polymyxin, gramicidin, cephalexin, oxytetracycline, chloramphenicol, kanamycin, rifampicin, tobramycin, gentamicin, erythromycin and penicillin; antibacterials such as sulfonamides, sulfadiazine, sulfacetamide, sulfamethizole and sulfisoxazole, nitrofurazone and sodium propionate; antivirals including idoxuridine, trifluorothymidine, acyclovir, ganciclovir and interferon; anti-allergenics such as sodium cromoglycate, antazoline, methapyriline, chlorpheniramine, cetirizine and prophenpyridadine; anti-inflammatories such as hydrocortisone, hydrocortisone acetate, dexamethasone, dexamethasone 21-phosphate, fluocinolone, medrysone, prednisolone acetate, fluoromethalone, betamethasone, and triamcinolone and non-steroidal agents such as indomethacin, diclofenac, flurbiprofen, piroxicam, ibuprofen and acetyl salicylic acid; decongestants such as phenylephrine, naphazoline and tetrahydrozoline: miotics and anticholinesterase such as pilocarpine, acetylcholine chloride, physostigmine, eserine, carbachol, di-isopropyl fluorophosphate, phospholine iodine, and demecarium bromide; mydriatics such as atropine sulfate, cyclopentolate, homatropine, scopolamine, tropicamide, eucatropine, and hydroxyamphetamine; sympathomimetics such as epinephrine; immunological drugs such as vaccines and immune stimulants; hormonal agents such as estrogens, estradiol, progestational, progesterone, insulin, calcitonin, parathyroid hormone and peptide, vasopressin, hypothalamus releasing factor; beta adrenergic blockers such as timolol maleate, levobunolol HCl and betaxolol HCl; growth factors such as epidermal growth factor and fibronectin; carbonic anhydrase inhibitors such as dichlorphenamide, acetazolamide and methazolamide and other drugs such as prostaglandins, antiprostaglandins, and prostaglandin precursors.

In a preferred embodiment of this invention, the ophthalmic drug is selected from the group consisting of pilocarpine, epinephrine, tetracycline, phenylephrine, eserine, phospholine iodide, demecarium bromide, cyclopentolate, homatropine, scopolamine, chlortetracycline, bacitracin, neomycin, polymixin, gramicidin, oxytetracycline, chloramphenicol, gentamycin, penicillin, erythromycin, carbachol, sulfacetamide, polymixin B, idoxuridine, isoflorophate, fluoromethalone, dexamethasone, hydrocortisone, hydrocortisone acetate, dexamethasone 21-phosphate, fluorocinolone, medrysone, prednisolone, methyl prednisolone, prednisolone 21-phosphate, prednisolone acetate, betamethasone, triamcinolone, or combinations thereof.

In a more preferred embodiment of this invention, the ophthalmic disorder is glaucoma or elevated intraocular pressure and the ophthalmic drug contained in the sustained-release pharmaceutical composition is an anti-glaucoma agent selected from the group consisting of pilocarpine, timolol, betaxolol, levobunolol, latanoprost, dorzolamide, epinephrine, dipivalyl epinephrine, brimonidine, or combinations thereof.

According to this invention, the sustained-release pharmaceutical composition may be administered into the mammal's eye via anterior chamber injection, intra-retinal injection, subretinal injection, intravitreal injection, or suprachoroidal injection, preferably using a 30 gauge needle.

When administering to a target site inside a mammal via injection, due to the thermo-responsive property of the hydrogel-forming polymer, the sustained-release pharmaceutical composition of this invention rapidly forms in situ a drug-incorporated hydrogel at or near the target site of said mammal, which provides an extended duration of action of the therapeutic agent to the mammal. As such, the hydrogel-forming polymer of this invention improves the therapeutic agent's bioavailability, so that the dosing frequency of the therapeutic agent could be reduced.

The dosage of the sustained-release pharmaceutical composition of this invention varies depending on the age, body weight, medical condition and drug response of the mammal, the unit dosage form, the administration frequency and the like. Preferably, a mammal is administered with the sustained-release pharmaceutical composition of this invention at a dose of 0.5 to 2.5 mg, and more preferably at a dose of 1 to 2 mg, at a dosing interval of 14 days or longer.

This invention will be further described by way of the following examples. However, it should be understood that the following examples are solely intended for the purpose of illustration and should not be construed as limiting the invention in practice.

### EXAMPLES

### Materials

Gelatin (type A, 300 Bloom, from porcine skin, weight average MW=˜100,000), 1-ethyl-3-(3-dimethyl aminopropyl) carbodiimide hydrochloride (EDC), adipic acid dihydrazide (ADH), ninhydrin reagent (2% solution), matrix metalloproteinase-2 (MMP-2, EC 3.4.24.24, synonym: gelatinase A), pilocarpine nitrate, and α-chymotrypsin were purchased from Sigma-Aldrich (St. Louis, Mo., USA). 2,2′-azobisisobutyronitrile (AIBN) was purchased from Otsuka Chemical (Tokyo, Japan). 1-hydroxybenzotriazole hydrate (HOBt) was purchased from Chem-Impex International (Wood Dale, Ill., USA). Sodium dodecyl sulfate (SDS) was purchased from Merck (Whitehouse Station, N.J., USA). 2-(N-morpholino)ethanesulfonic acid (MES) and dimethyl sulfoxide (DMSO) were purchased from J. T. Baker (Phillipsburg, N.J., USA).

N-isopropylacrylamide (NIPAAm) and N-hydroxysuccinimide (NHS) were purchased from Acros Organics (Geel, Belgium). Prior to use, NIPAAm and AIBN were purified by recrystallization from n-hexane and methanol, respectively. Mercaptoacetic acid (MAA) supplied by Nacalai Tesque (Kyoto, Japan) was purified by distillation under reduced pressure.

Deionized water used was purified with a Milli-Q system (Millipore, Bedford, Mass., USA). MES was dissolved in deionized water to form a 0.1M MES buffer solution (pH 5.0). Balanced salt solution (BSS, pH 7.4) was purchased from Alcon Laboratories (FortWorth, Tex., USA). Phosphate-buffered saline (PBS, pH 7.4) was purchased from Biochrom (Berlin, Germany). Dulbecco's modified Eagle's medium (DMEM), Eagle's minimum essential medium (MEM), and TRIzol reagent were purchased from Gibco-BRL (Grand Island, N.Y., USA). Fetal bovine serum (FBS) and the antibiotic/antimycotic (NA) solution (10,000 U/mL penicillin, 10 mg/mL streptomycin and 25 mg/mL amphotericin B) were purchased from Biological Industries (Kibbutz Beit Haemek, Israel).

All the other chemicals were of reagent grade and used as received without further purification.

### General Experimental Procedures:

### 1. Proton Nuclear Magnetic Resonance Spectroscopy (¹H-NMR)

The ¹H-NMR analysis was performed using a Bruker Avance DRX 500 NMR instrument at a 500 MHz frequency (Taipei Medical University, Taipei, Taiwan, ROC). The ¹H-NMR spectra were recorded using an internal deuterium lock at ambient temperature. The ¹H chemical shift scale was referenced against internal DMSO-86 at 2.50 ppm.

### 2. Fourier Transform Infrared Spectroscopy (FTIR)

The FTIR analysis was performed using a FT-730 ATR-FTIR Spectrophotometer (Horiba, Japan) according to the method reported in David Hui-Kang Ma et al. (2010), Biomaterials, 31(25):6647-6658. The FTIR spectra of test samples were recorded between 3800 and 800 cm⁻¹ with a resolution of 8 cm⁻¹. The data were analyzed using the program FTIR Spectrum software (Horiba, Japan). The experiment was performed in triplicate, and the results were averaged.

### 3. Statistics

Results were expressed as mean±standard deviation. Comparative studies of mean values were performed using one-way analysis of variance (ANOVA). Significance was accepted with P<0.05.

### Synthesis Examples

### Experimental Procedures

A. Synthesis of a Thermo-Responsive Component According to this Invention

This section illustrates the production of a carboxylic end-capped poly(N-isopropylacrylamide), which was used as a thermo-responsive component according to this invention in the following examples and experiments.

A carboxylic end-capped poly(N-isopropylacrylamide) (PNIPAAm-COOH), which was referred to as “PN” hereinafter, was synthesized by free radical polymerization technique. Briefly, 50 g of NIPAAm, 0.36 g of AIBN, and 3.07 mL of MAA (equivalent to a MAA/NIPAAm molar ratio of 0.1) were added into a four-necked flask and dissolved in 250 mL of benzene. The resultant mixture was bubbled with nitrogen gas for 10 min and then stirred (300 rpm) at 60° C. for 24 hours under nitrogen. After polymerization, the solvent (benzene) was removed by evaporation in a chemical hood, and the residue was dissolved in acetone and purified by re-precipitation with diethyl ether. The precipitated product was re-dissolved in deionized water and then exhaustively dialyzed (MWCO: 3,500, Spectra/Por® Dialysis Membrane, Rancho Dominguez, Calif., USA) against deionized water at 4° C. for 7 days. The product thus purified was lyophilized at −50° C. and kept in a closed vessel at room temperature prior to use.

The number-average molecular weight (Mn) of the purified product (i.e., PN) was determined by end group titration as described in Jyh-Ping Chen et al. (2006), Macromol. Biosci., 6:1026-1039. Briefly, 0.1 g of a test sample was dissolved in 10 mL of deionized water and the resultant solution was titrated with 0.01 N NaOH to determine the concentration of carboxylic end group. The Mn of PN (MW_{PN}) was calculated according to the following equation (1). The experiment was performed in triplicate, and the results were expressed as mean±SD.

$$M {W}_{PN} = \frac{weight of PN titrated}{moles of carboxylic end groups of PN} \qquad ( 1 )$$

B. Synthesis of a Biodegradable Component According to this Invention

This section illustrates the production of an aminated gelatin, which was used as a biodegradable component according to this invention in the following examples and experiments.

An aminated gelatin, which was referred to as “A-gelatin” hereinafter, was synthesized according to the protocol used for preparation of aminated hyaluronic acid (AHA) as reported in Huaping Tan et al. (2009), Biomaterials, 30(36):6844-6853, except for some modifications. Briefly, 1 g of gelatin and 2.36 g of ADH were dissolved in 200 mL of deinonized water to form an aqueous solution. Meanwhile, 2.79 g of EDC and 1.83 g of HOBt were dissolved in DMSO/H₂O (1:1 v/v, 6.5 mL each), followed by addition into the aqueous solution. The resultant mixture was adjusted to a pH of 5.0 with 1N HCl and then stirred (100 rpm) at 25° C. for 24 hours. Thereafter, the reaction mixture was exhaustively dialyzed (MWCO: 3,500, Spectra/Por® Dialysis Membrane, Rancho Dominguez, Calif., USA) against deionized water for 3 days. To the dialyzed solution was added NaCl to a concentration of 5% w/v, followed by precipitation in ethanol. The resultant precipitate was dissolved in deionized water, followed by dialysis for another 3 days to remove the salt. The product thus purified was lyophilized at −50° C. and kept at 4° C. prior to use.

To verify the successful amination of gelatin by ADH, a sample of the purified product was subject to the ¹H-NMR analysis as described in the preceding section of General Experimental Procedures.

The amount of free amino groups in the purified product was determined by a ninhydrin assay as described in Jui-Yang Lai and Ya-Ting Li (2010), Materials Science and Engineering C, 30:677-685. Briefly, 2 mg of a test material was evenly admixed with 2 mL of an aqueous acetic acid solution (0.05%, w/v) and 1 mL of the ninhydrin reagent in dark, followed by heating in a 100° C. water bath for 20 minutes. Thereafter, the reaction mixture was cooled down at room temperature for 15 minutes and then diluted with 5 mL of 95% ethanol. The optical absorbance of a test solution thus prepared was recorded with a UV-visible spectrophotometer (Thermo Scientific, Waltham, Mass., USA) at 570 nm using glycine at various known concentrations as standard. The amount of free amino groups in the test material is proportional to the detected optical absorbance of the test solution. Results from five independent experiments were averaged.

To determine the molecular weight of the purified product, the five ninhydrin-treated solutions were combined, vortexed, and pipetted into a syringe for filtration (0.45 μm filter, Millipore), followed by gel permeation chromatography (GPC) using a system consisting of a HPLC-pump and a RI 2000 refractive index detector (Schambeck SFD GmbH, Bad Honnef, Germany) with four thermostated (37° C.) columns (Shodex SB series, OHpak SB-802 HQ (exclusion limit 4×10³ Da); OHpak SB-802.5 HQ (exclusion limit 1×10⁴ Da); OHpak SB-803 HQ (exclusion limit 1×10⁵ Da); OHpak SB-804 HQ (exclusion limit 1×10⁶ Da); Showa Denko, Tokyo, Japan). The mobile phase was 18 g/L SDS in Milli-Q water. The flow rate was 1 mL/min. Poly(ethylene glycol) (PEG) and poly(ethylene oxide) (PEO) standards (Polymer Standards Service, Mainz, Germany) ranging between 434 and 932000 g/mol were used for calibration. The chromatograms were analyzed with Analab EC2000 Data System. Mean peak molecular weight (Mw) in kDa was calculated.

C. Synthesis of a Hydrogel-Forming Polymer According to this Invention

This section illustrates the production of a hydrogel-forming polymer gelatin-g-PNIPAAm according to this invention, which was formed from the grafting reaction of PN (i.e., the product obtained in the preceding section A) and A-gelatin (i.e., the product obtained in the preceding section B).

A hydrogel-forming polymer gelatin-g-PNIPAAm, which was referred to as “GN” hereinafter and used in the following examples and experiments, was synthesized as follows: 10 g of PN (i.e., the product as obtained in the preceding section A) was dissolved in 50 mL of MES buffer containing 2.59 g of EDC and 1.55 g of NHS under agitation for 6 hours, followed by addition of 50 mL of MES buffer containing 1 g of A-gelatin (i.e., the product as obtained in the preceding section B). After reacting at 25° C. for 24 hours with stirring (100 rpm), the reaction mixture was placed in a thermostatic bath set at 50° C. for 30 minutes, followed by centrifugation at 9,000 rpm for 20 minutes. The collected precipitate was resuspended in deionized water and then exhaustively dialyzed (MWCO: 50,000, Spectra/Por® Dialysis Membrane, Rancho Dominguez, Calif., USA) against deionized water at 4° C. for 4 days. The product thus purified was lyophilized at −50° C. and kept in a closed vessel at room temperature.

A sample of the purified product, as well as gelatin, A-gelatin, NIPAAm and PN, was subject to the FTIR analysis as described in the preceding section of General Experimental Procedures.

The grafting ratio (GR), efficiency of grafting (EG), degree of grafting (DG), and weight ratio of PN to A-gelatin (P/G) for the purified product (i.e., GN) were determined according to the following equations (2)˜(5), respectively (Jyh-Ping Chen et al. (2006), Macromol. Biosci., 6:1026-1039; Shoji Ohya and Takehisa Matsuda (2005), J. Biomater. Sci. Polymer Edn., 16(7):809-827):

$$\begin{matrix}GR = \frac{( {W}_{GN} - {W}_{G} ) / M {W}_{PN}}{{W}_{G} / M {W}_{G}} & ( 2 ) \\ EG = \frac{{W}_{GN} - {W}_{G}}{{W}_{PN}} \times 100 & ( 3 ) \\ DG = \frac{GR}{n} \times 100 & ( 4 ) \\ P / G = \frac{( {W}_{GN} - {W}_{G} )}{{W}_{G}} & ( 5 )\end{matrix}$$

wherein:

W_{GN} represents the weight of the purified product;

W_{G} and W_{PN} represent the weights of A-gelatin and PN used in the reactions, respectively;

MW_{G} and MW_{PN} represent the molecular weights of A-gelatin and PN, respectively; and

n is the number of amino groups in the peptide chain of A-gelatin as determined by the ninhydrin assay.

### D. Phase Transition Characterizations

To investigate the influence of polymer concentration on the thermally induced morphological change, a sample solution containing 2.5 or 10% w/v of a test material (PN obtained in the preceding section A, A-gelatin obtained in the preceding section B, or GN obtained in the preceding section C) was prepared by dissolving said test material in deionized water. The morphological change of the sample solution was visually observed during heating from 25 to 34° C.

The phase transition property of each test sample (PN obtained in the preceding section A, and GN obtained in the preceding section C) was investigated using a DSC 2010 differential scanning calorimeter (TA Instruments, New Castle, Del., USA). The test sample was dissolved in deionized water or BSS to a concentration of 10% (w/v). After equilibration at room temperature for 1 hour, the sample solutions (each 8 mg) were hermetically sealed in aluminum pans for DSC experiment. Programmed heating was carried out at 3° C./min in the temperature range from 25° C. to 45° C., under a nitrogen gas flow. The lower critical solution temperature (LCST) was determined as the onset point of the endothermic peak (Xian-Zheng Zhang et al. (2001), Langmuir, 17, 6094-6099). Results from five independent experiments were averaged.

The absorbance change of each test sample (PN obtained in the preceding section A, and GN obtained in the preceding section C) due to thermally induced sol-gel phase transition was detected by a UV-visible spectrophotometer (Thermo Scientific, Waltham, Mass., USA). The test sample was dissolved in deionized water or BSS to a concentration of 10% (w/v). After incubation at 25° C. for 1 hour, the sample solutions were placed in a thermostatic cell holder that had been pre-equilibrated at 34° C. The time-course of the optical absorbance change of each sample solution was measured at 470 nm. The gel formation time (GFT) was determined as the time at which the absorbance reached half of the difference between the maximum and minimum values (Jyh-Ping Chen et al. (2006), Macromol. Biosci., 6:1026-1039). Results from five independent experiments were averaged.

### Results and Discussion:

### 1. Synthesis of Carboxylic End-Capped Poly(N-Isopropylacrylamide)

In addition to 4,4′-azobis(4-cyanovaleric acid) (Huaping Tan et al. (2009), Biomaterials, 30(36):6844-6853), AIBN is a commonly used initiator in free radical polymerization of carboxylic acid-terminated PNIPAAm (Jyh-Ping Chen et al. (2006), Macromol. Biosci., 6:1026-1039; Sevil Dincer et al. (2002), Macromol. Chem. Phys., Vol. 203, No. 10/11, pp. 1460-1465). In the study of this invention, the applicant synthesized a thermo-responsive component having a single carboxylic acid group at one end of the molecule thereof from the polymerization reaction of NIPAAm monomer in the presence of AIBN and MAA (a chain transfer agent having a carboxyl group in the molecule thereof). Based on the end group titration results (approximately 135 μmoles of carboxylic end groups per gram of the purified product) and equation (1), the carboxylic end-capped poly(N-isopropylacrylamide) synthesized in the preceding section A of the Experimental Procedures, i.e., PN, was calculated to have a number-average molecular weight (Mn) of 7407±570 Da (corresponding to a polymerization degree of 65).

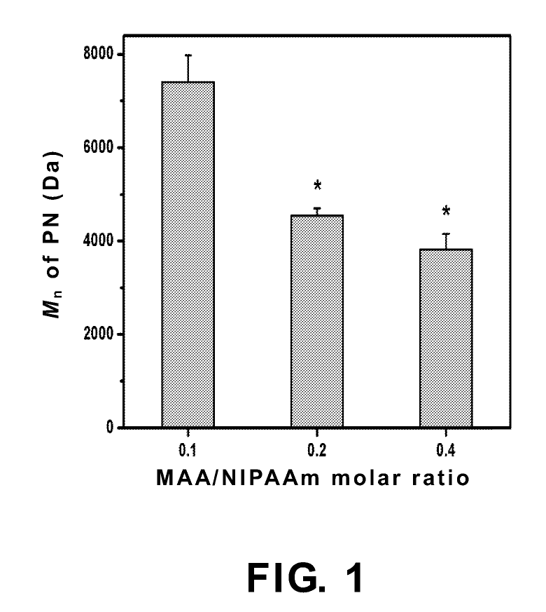

The effect of the molar ratio of MAA/NIPAAm on the Mn of the synthesized carboxylic end-capped poly(N-isopropylacrylamide) was also studied. Referring to FIG. 1, in the range from 0.1 to 0.4, the Mn significantly decreased with an increasing MAA/NIPAAm molar ratio (P<0.05), probably due to the fact that a high MAA concentration may lead to early chain termination and a low molecular weight (Jyh-Ping Chen et al. (2006), Macromol. Biosci., 6:1026-1039).

Ohya et al. have demonstrated that the higher molecular weight of PNIPAAm graft chain can induce more obvious gel formation, thereby is beneficial to fabricate an in situ gellable or injectable three-dimensional artificial extracellular matrix (Shoji Ohya (2005), J. Biomater. Sci, Polymer Edn., 16(7):809-827). Furthermore, it has been reported that the low-molecular-weight fraction of proteins (<10 kDa) in aqueous humor has little or no blocking effect, whereas the high molecular-weight fraction of proteins obstructs the outflow of the fluid from the eye (C. Ross Erhier et al. (1989), Invest. Ophthalmol. Vis. Sci., 30(4):739-746).

Based on these considerations, the PN synthesized in the preceding section A, which had a Mn of 7407±570 Da and a polymerization degree of 65, was used for grafting onto the amino functional groups of the biodegradable component of this invention.

### 2. Synthesis of Aminated Gelatin

Since gelatin is an amphiphilic polyelectrolyte, the formation of cross-links (i.e., amide bonds) between the free amino and carboxylic groups in the polypeptide chain usually occurs during EDC/NHS coupling of the biodegradable and thermo-responsive polymers (Jui-Yang Lai and Ya-Ting Li (2011), Journal of Biomaterials Science, 22:277-295). To reduce this inevitable reaction, in the study of this invention, gelatin was aminated with ADH to increase its free amine functionality. According to the ninhydrin assay results, the amount of amino group of lysine residue in gelatin was evaluated to be 33.1±0.1 per molecule, which is in good agreement with that reported in the literature (Nobuyuki Morikawa et al. (2002), J. Biomater. Sci. Polymer Edn., 13(2):167-183). After amination with ADH, the amount of amino group in A-gelatin obtained in the preceding section B of the Experimental Procedures was significantly increased to 48.7±0.4 per molecule (P<0.05).

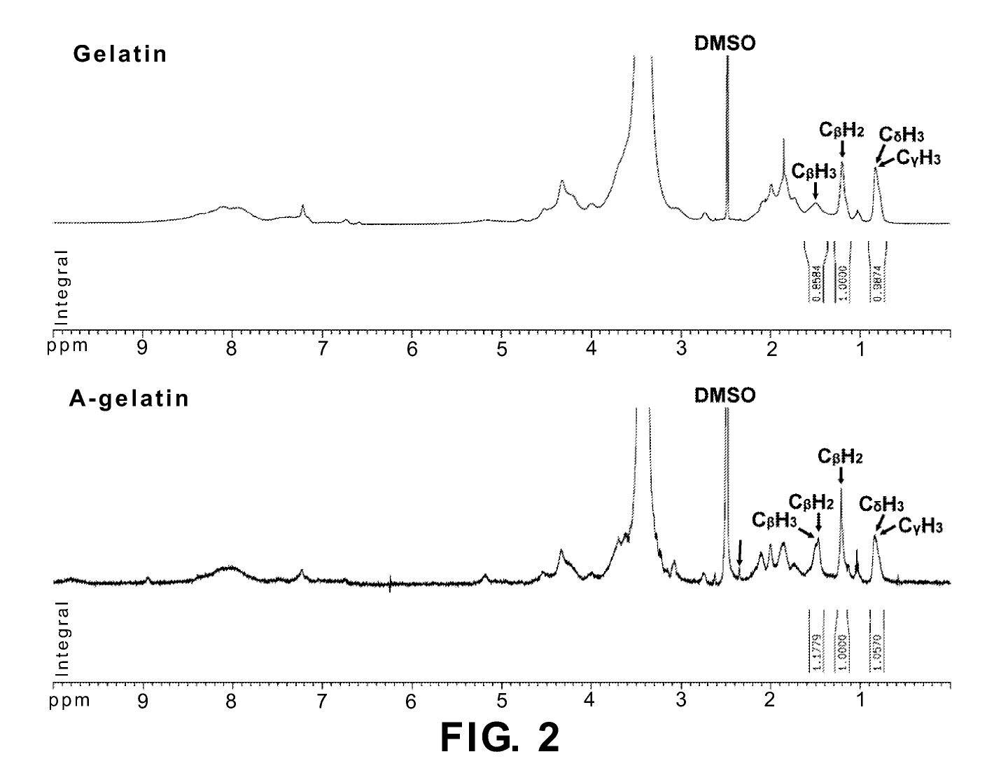

Gelatin and A-gelatin were further subjected to ¹H-NMR analysis. Referring to FIG. 2, a list of chemical shift assignments for the gelatin is given as follows: 1.56 ppm for alanine C_{β}H₃, 1.22 ppm for proline C_{β}H₂, and 0.8 ppm for valine C_{γ}H₃ and leucine C_{δ}H₃ (Andrew R. Pickford et al. (1997), Structure, 5:359-370). In Biomaterials (2009), 30(36):6844-6853, Huaping Tan et al. reported that for the aminated hyaluronic acid, the peaks between 2.2 and 2.4 ppm referred to the integration of methylene protons of ADH. In addition, a peak at 1.5 ppm is assigned to the ADH C_{β}H₂. In accordance with these earlier observations, the spectral data of A-gelatin shown in FIG. 2 reveal the appearance of a peak at 2.35 ppm and an increased integration of the characteristic peak at 1.5 ppm, indicating the successful amination of gelatin by ADH.

Furthermore, according to the GPC analysis results, gelatin and A-gelatin were determined to have a molecular weight (Mw) of approximately 99 and 102 kDa, respectively. The slight increase in the molecular weight of A-gelatin may reflect the binding of ADH to gelatin without protein self-crosslinking.

### 3. Synthesis of Gelatin-g-PNIPAAm

To eliminate the risk of bovine spongiform encephalopathy from bovine-based gelatin, the materials produced by an acidic processing of porcine skin collagen were recently fabricated as porous hydrogel carriers for corneal endothelial cell delivery (Jui-Yang Lai and Ya-Ting Li (2010), Biomacromolecules, 11(5):1387-1397). In the study of this invention, porcine-based gelatins were used as backbone networks in the synthesis of polymeric brushes exhibiting temperature-dependent response.

The hydrogel-forming polymer gelatin-g-PNIPAAm (i.e., GN) was synthesized by reacting 10 g of PN (estimated to have 1350 μmol of COOH groups) with 1 g of A-gelatin (estimated to have 487 μmol of NH₂ groups) in the presence of EDC/NHS coupling reagents.

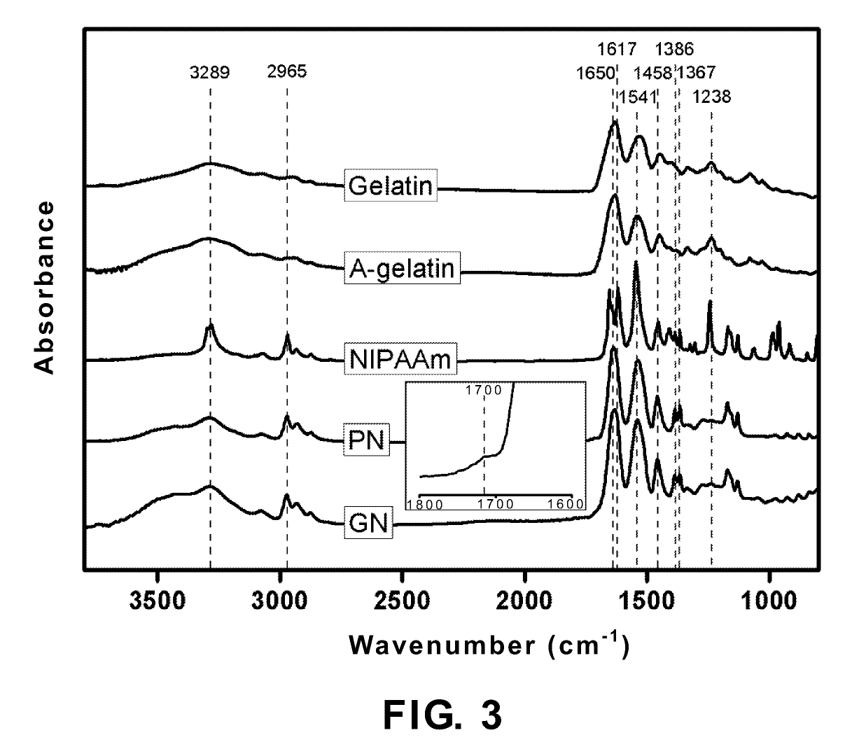

FIG. 3 shows the FTIR spectra of gelatin, A-gelatin, NIPAAm, PN and GN. Referring to FIG. 3, gelatin has several absorption bands at 3289 cm⁻¹ (N—H stretching vibration), 2965 cm⁻¹ (C—H stretching vibration), 1650 cm⁻¹ (amide I, C═O stretching vibration), 1541 cm⁻¹ (amide II, N—H bending vibration), and 1238 cm⁻¹ (amide III, N—H bending vibration), which are typical of those observed for proteins (Jui-Yang Lai and Ya-Ting Li (2011), Journal of Biomaterials Science, 22:277-295). Although A-gelatin has a similar pattern of spectra, the peak intensities at these wavelengths slightly increased after amination of gelatin with ADH.

In addition to evident amide signals, the peaks at 1458, 1386 and 1367 cm⁻¹ were observed for both NIPAAm and PN, attributable to symmetric and antisymmetric deformation of —C(CH₃)₂, respectively (Changyou Gao et al. (2005), Polymer, 46:4088-4097).

After free radical polymerization of NIPAAm, the absorbance band at 1617 cm⁻¹ was not observed in the FTIR spectra of PN, indicating that the C═C bonds present in the NIPAAm monomers were broken to form the C—C bonds in the polymer backbone chain of PN. A very small peak at 1710 cm⁻¹ is observed in the FTIR spectra of PN, suggesting the successful synthesis of the thermo-responsive component having a single carboxylic acid group at one end of the molecule thereof.

In the study of this invention, GN was obtained by coupling A-gelatin with PN via a covalent bonding formed between one of the amino functional groups of A-gelatin and the carboxylic acid end group of PN. As such, the FTIR spectra of GN were observed to have the characteristic absorbance peaks of amides and —C(CH₃)₂.

In Biomacromolecules (2010), 11(9):2261-2267, Scott D. Fitzpatrick et al. reported that the PNIPAAm-grafted collagen can be synthesized by EDC/NHS chemistry. The findings of their study suggest that during the generation of covalent linkages between the carboxylic acid groups of aspartic acid and glutamic acid residues present in collagen with the amine functionalized end groups of the PNIPAAm, the slight collagen cross-linking may occur. Similarly, the possibility of gelatin crosslinking should not be excluded totally, although the carboxylic acid groups of PN are allowed to activate for 6 hours prior to reaction with A-gelatin in the presence of the same coupling reagents. To check the grafting reaction, GN was characterized by various parameters such as GR (i.e., the number of PN chains grafted onto an A-gelatin backbone molecule), EG (i.e., the percentage of grafted PN based on the initial amount of PN used in the reaction), DG (i.e., the percentage of amino groups in the A-gelatin backbone used for the grafting reaction), and P/G (i.e., the weight ratio of PN grafted chains to A-gelatin in GN). In the range from 2.59 to 12.93, the grafting effectiveness for the PNIPAAm-grafted chitosan decreases with increasing feed molar ratio of NH₂/COOH (Jyh-Ping Chen et al. (2006), Macromol. Biosci., 6:1026-1039). However, according to the applicants' experience, the GN samples prepared at a NH₂/COOH molar ratio greater than 1 exhibit poor thermo-responsiveness, implying that the grafting yield is relatively low (data not shown). Hence, to confer the hydrogel-forming polymer of this invention with rapid in situ gelling ability that imparts better delivery capabilities, in the study of this invention, the feed molar ratio of NH₂/COOH was controlled at 0.36, resulting in the gelatin-g-PNIPAAm (i.e., GN) thus formed to have GR=25.6, EG=18.6%, DG=52.6%, and P/G=1.9.

### D. Phase Transition Characterizations

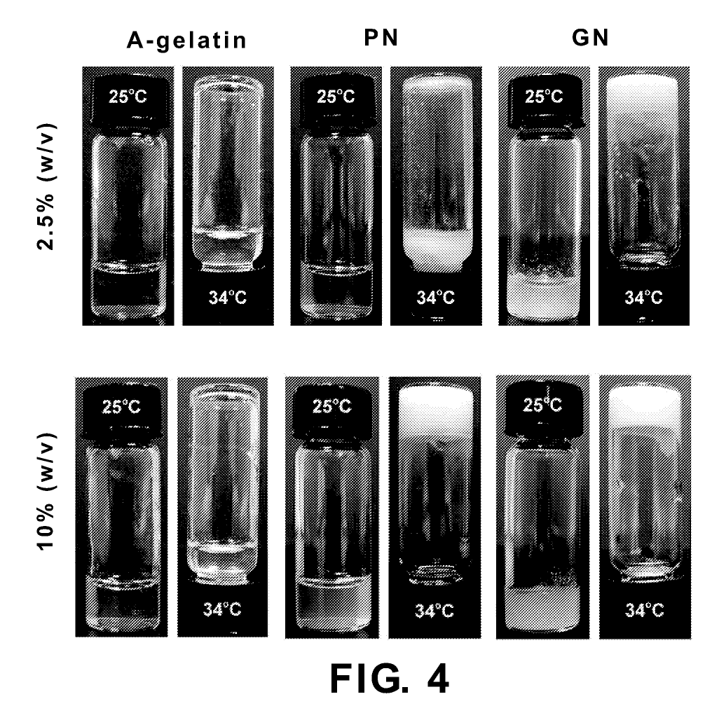

It is known that the external temperature variations can change the hydrophilic-hydrophobic balance of a thermo-responsive polymer, resulting in a phase transition (Xiao-Ding Xu et al. (2008), J. Biomed. Mater. Res. A, 86(4):1023-1032). To investigate the thermo-responsive properties of the hydrogel-forming polymer of this invention, the morphological changes of A-gelatin, PN and GN were visually observed during heating from 25° C. to 34° C. The gross morphological observation results shown in FIG. 4 reveal that irrespective of their concentration, the A-gelatin solutions did not undergo morphological changes during the heating from 25 to 34° C. However, the temperature triggered sol-gel phase transitions occurred for both the PN and GN solutions. When the polymer concentration was low (i.e., 2.5% w/v) (FIG. 4, upper panel), the PN matrices formed at 34° C. were fragile and unable to adhere to the bottom of the vial upon inversion, whereas the GN gels formed at 34° C. exhibited remarkable adherence properties due to the viscosity building effects of gelatin (Jui-Yang Lai (2009), Int. J. Mol. Sci., 10:3442-3456). This finding may suggest that GN has potential for application as an in situ hydrogel-forming delivery system. At higher polymer concentration (i.e., 10% w/v), the sol-gel phase transitions were more complete with decreased solution flow, indicating enhancement of thermal gelation ability (FIG. 4, lower panel).

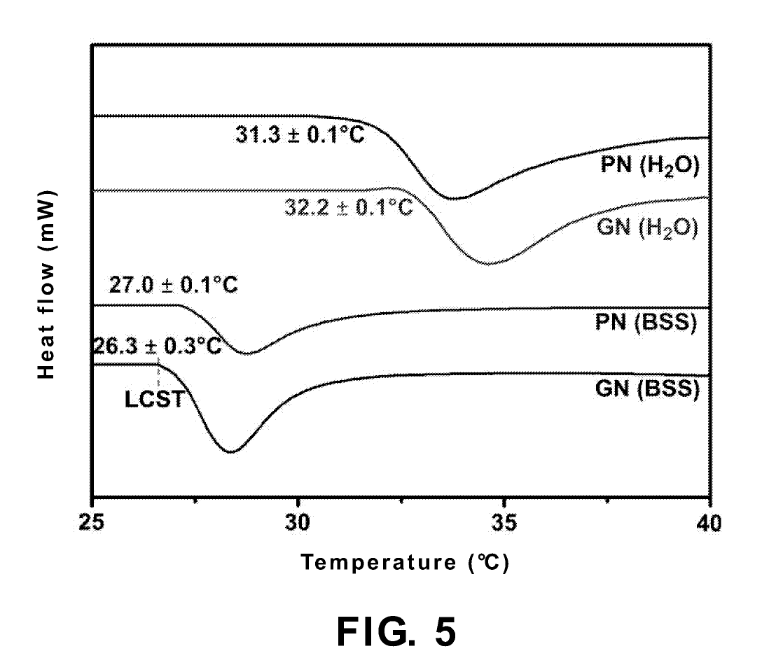

FIG. 5 shows the DSC thermograms of PN and GN in deionized water or BSS. At temperatures above the LCST, the hydrophobic interaction becomes the dominating force for the aggregation of macromolecules in solution because of the thermal dissociation of hydrating water molecules from the polymer chains. When dissolved in deionized water, PN and GN were determined to have a LCST of 31.3±0.1° C. and 32.2±0.1° C. (n=5), respectively.

According to the synthesis procedures set forth in the preceding section

A of Experimental Procedures, the applicants synthesized a second hydrogel-forming polymer using gelatin in place of A-gelatin, and the second hydrogel-forming polymer thus obtained was determined to have a LCST of 32.2±0.2° C. (n=5) in deionized water, which is comparable to that of GN.

Several reports have shown that the incorporation of a hydrophilic moiety such as acrylic acid (Brent Vernon et al. (2000), J. Biomed. Mater. Res., 51:69-79) and ethylene glycol (Dirk Schmaljohann et al. (2003), Biomacromolecules, 4(6):1733-1739) into PNIPAAm results in a higher LCST. In the study of this invention, the applicants found that the gelatin network led to an increase in the transition temperature of the PNIPAAm-containing hydrogel-forming polymer in deionized water.

In order to realize the thermal response of the hydrogel-forming polymer of this invention under physiological conditions, same DSC experiments were performed using BSS buffer, which contains various salts. Referring to Table 1, the LCSTs of PN and GN in BSS were 4.3 and 5.9° C. less than those in deionized water, respectively, probably due to the salting out effect (Xue-Ming Liu et al. (2004), Biomaterials, 25:5659-5666). Additionally, it is of interest to note that an inverse result on the LCST of each test sample was observed when dissolved in BSS. These results indicate that the salt may exhibit a stronger effect on the LCST of a hydrogel-forming polymer constituted of an amphiphilic peptide sequence grafted with PNIPAAm chains.

TABLE 1
The LCST and phase transition data for PN and GN

| | LCST (° C.) | | Gel formation time (sec) | |
|---|---|---|---|---|
| Polymer^{a} | H₂O | BSS | H₂O | BSS |
| PN | 31.3 ± 0.1^{b} | 27.0 ± 0.1 | 23.1 ± 1.2 | 19.8 ± 1.4 |
| GN | 32.2 ± 0.1 | 26.3 ± 0.3 | 26.2 ± 1.0 | 15.7 ± 1.1 |

^{a}Concentration of the test polymer = 10% (w/v).
^{b}For each experiment, n = 5.

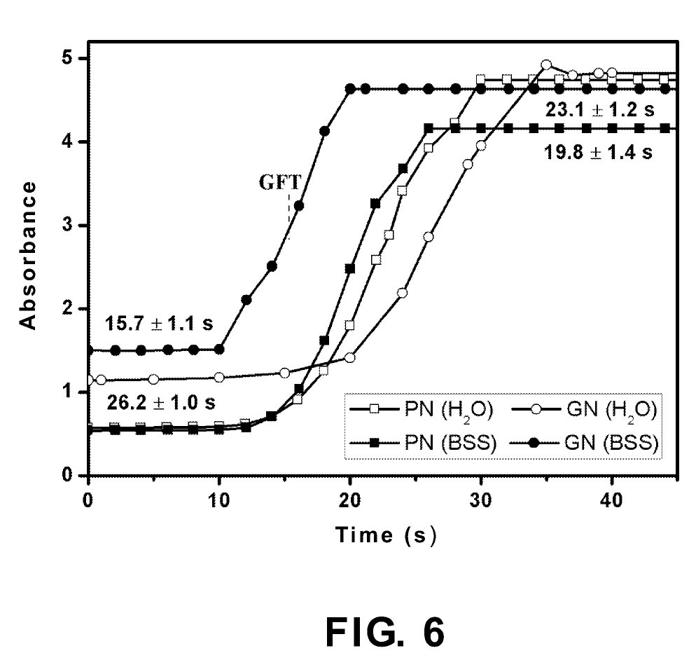

The phase transition kinetics of temperature-sensitive polymers may be recorded by time-course UV spectroscopy. Referring to FIG. 6 and Table 1, during heating from 25 to 34° C., a sudden increase in absorbance occurred for GN dissolved in BSS, demonstrating that GN gelled in BSS more quickly than in H₂O. When dissolved in deionized water, PN was determined to have a gel formation time (GFT) of 23.1±1.2 sec, which was significantly lower than that of GN (26.2±1.0 sec) (P<0.05). A possible explanation is that the aggregation of the PN chains in aqueous media is slightly influenced by the steric hindrance of the GN polymers. In contrast, when dissolved in BSS, GN exhibited a shorter GFT than that of PN, suggesting that the presence of salt may accelerate the phase transition of a polymeric system containing amphiphilic moieties. These findings are consistent with those obtained from the LCST measurements.

Recently, injectable in situ forming biodegradable hydrogels have been explored for various tissue engineering approaches including adipose (Huaping Tan et al. (2009), Biomaterials, 30(36):6844-6853), cartilage (Huaping Tan et al. (2009), Biomaterials, 30(13):2499-2506), cardiac (Kazuro L. Fujimoto et al. (2009), Biomaterials, 30(26):4357-4368), and retinal (Scott D. Fitzpatrick et al. (2010), Biomacromolecules, 11(9):2261-2267) regeneration. These biomaterial matrices may allow graft delivery with minimal surgical intervention and provide temporary support for cell retention and distribution at the site of injury. The bioactive gelling scaffolds also establish a microenvironment that facilitates the proliferation and organization of encapsulated cells. Because of their dual function in utilization as carrier system, the PNIPAAm-chitosan copolymers have been shown to have potential for preparation of controlled release ophthalmic drops (Yanxia Cao et al. (2007), J. Controlled Release, 120(3):186-194). In earlier studies, the applicants used PNIPAAm and gelatin to design a minimally invasive strategy for corneal endothelial reconstruction that can reduce the risk of suture-related complications (Ging-Ho Hsiue et al. (2006), Transplantation, 81(3):473-476; Jui-Yang Lai et al. (2006), Transplantation, 84(10):1222-1232). However, to the best of the applicants' knowledge, biodegradable in situ forming gels as injectable intraocular drug delivery systems (DDSs) for glaucoma therapy has yet to be available.

In view of the experimental results of GN as described above, GN was expected to have potential as a carrier in hydrogel-type sustained-release drug delivery systems. Therefore, GN was further evaluated to determine its usefulness in improving the therapeutic efficacy of pilocarpine after intracameral administration in glaucomatous rabbits.

### Pharmacological Experiments

### A. In Vitro Degradation Test

To evaluate the biodegradability of hydrogel-forming polymers, a test sample containing 10% w/v of a test material (i.e., PN or GN as obtained in the preceding section A or C of the Experimental Procedures of the Synthesis Example, respectively) was prepared by dissolving said test material in deionized water at 25° C., followed by transferring to a 34° C. thermostatically controlled water bath for 10 min to allow gelation. Thereafter, the hydrogel formed in the test sample was dried to constant weight (Wᵢ) in vacuo and then immersed at 34° C. in a degradation medium (BSS containing 50 ng/mL MMP-2). The degradation medium was replaced weekly. At designated time intervals, the MMP-2-treated hydrogel was collected and further dried in vacuo. The dry weight of the collected hydrogel after MMP-2 treatment (W_{d}) was measured and the percentage of weight remaining (%) was calculated as (W_{d}/Wᵢ)×100. Results from three independent measurements were averaged.

### B. In Vitro Biocompatibility Studies

The cellular responses to carrier materials were determined with a slight modification of the method described for cytotoxicity assessment of photopolymerized dextran and hyaluronan-based hydrogels using Transwell® inserts (J. Trudel and S. P. Massia (2002), Biomaterials, 23(16):3299-3307). In the study of this invention, a bovine corneal endothelial cell line BCE C/D-1b (ATCC CRL-2048) and a human lens epithelial cell line HLE-B3 (ATCC CRL-11421) purchased from the American Type Cell Collection (Manassas, Va., USA) were used in cytotoxicity assay. The BCE C/D-1b cells were maintained in regular growth medium containing DMEM, 10% FBS, 4 mM L-glutamine, 1.5 mg/mL sodium bicarbonate, 4.5 mg/mL glucose, and 1% NA solution. The HLE-B3 cells were grown in MEM supplemented with 20% FBS, 2 mM L-glutamine, 1 mM sodium pyruvate, 0.1 mM nonessential amino acids, 1.5 mg/mL sodium bicarbonate, and 1% NA solution. All of the cell cultures were incubated in a humidified atmosphere of 5% CO₂ at 37° C.

1 mL of a cell culture (either BCE C/D-1b or HLE-B3) was seeded into one well of a 24-well plate at a cell density of 5×10⁴ cells/mL and then incubated overnight to allow cell attachment. Thereafter, the medium in said well was replaced with fresh culture medium. Using a cell culture insert (Falcon 3095, Becton Dickinson Labware, Franklin Lakes, N.J., USA), said well was divided into two compartments, and to the inner compartment thus formed was added 150 μL of a sterile PN or GN solution (10% w/v), resulting in the in situ formation of a hydrogel in the inner compartment. The cells cultured in said well were examined after 48 hour exposure to the in situ formed hydrogel. Cells cultured with culture medium only served as a control.

The qualitative and quantitative examinations of the cultured cells were performed subsequent to the removal of the cell culture insert and the in situ formed hydrogel.

Cell morphology was observed using a phase-contrast microscope (Nikon, Melville, N.Y., USA).

Cell viability was determined by a membrane integrity assay using the Live/Dead Viability/Cytotoxicity Kit (Molecular Probes, Eugene, Oreg., USA) which contains calcein AM and ethidium homodimer-1 (EthD-1)(Jui-Yang Lai et al. (2006), Arch Ophthalmol, 124:1441-1448). This assay depends on the intracellular esterase activity to identify the living cells, which cleaves the calcein AM to produce a green fluorescence. For dead cells, EthD-1 can easily pass through the damaged cell membranes to bind to the nucleic acids, yielding a red fluorescence. In practice, the cultured cells were washed three times with PBS and then stained with a working solution consisting of 2 mL of EthD-1, 1 mL of PBS and 0.5 mL of calcein AM, followed by visual observation under a fluorescence microscope (Axiovert 200M; Carl Zeiss, Oberkochen, Germany).

Cell growth was estimated using the CellTiter 96 Aqueous Non-Radioactive Cell Proliferation MTS Assay kit (Promega, Madison, Wis., USA), in which MTS tetrazolium compound is bio-reduced by cells to form a water-soluble colored formazan (Pei-Lin Lu et al. (2008), J. Biomater. Sci. Polymer Edn., 19(1):1-18). The amount of said colored product is proportional to the number of metabolically active cells. In practice, the cells cultured in each well of a 24-well plate was treated with 100 μL of a combined MTS/PMS (20:1) solution and then incubated at 37° C. in a CO₂ incubator for 3 hours, followed by measuring absorbance at 490 nm using the Multiskan Spectrum Microplate Spectrophotometer (ThermoLabsystems, Vantaa, Finland). The experiment was performed in quadruplicate, and the results were expressed as relative MTS activity as compared to the control group (i.e., cells cultured with culture medium only).

The pro-inflammatory cytokine interleukin-6 (IL-6) expression of the cultured cells was detected at messenger RNA (mRNA) levels (Jui-Yang Lai (2009), Int. J. Mol. Sci., 10(8):3442-3456). Briefly, total RNA was isolated from the cultured cells with TRIzol reagent according to the manufacturer's procedure. Reverse transcription of the extracted RNA (1 μg) was performed using ImProm-II (Promega) and Oligo(dT)15 primers (Promega). The primer pairs used in detecting the gene expression of bovine and human IL-6 genes are listed in Table 2. Quantitative real-time reverse transcription polymerase chain reaction (RT-PCR) was performed on a Light-Cycler instrument (Roche Diagnostics, Indianapolis, Ind., USA) according to the manufacturer's instructions with FastStart DNA Master SYBR Green I reagent (Roche Diagnostics). The mRNA expression of IL-6 was normalized with that of glyceraldehyde 3-phosphate dehydrogenase (GAPDH). The experiment was performed in triplicate, and the results were expressed as relative IL-6 mRNA level (%) as compared to the control group (i.e., cells cultured with culture medium only).

TABLE 2
Primer pairs used in detecting the gene
expression of bovine and human IL-6 genes.

| Genes | Primer | Nucleotide Sequence (5′→3′) |
|---|---|---|
| Bovine | Forward | ccaccccaggcagactacttc |
| IL-6 | | (SEQ ID NO: 1) |
| | Reverse | ctccttgctgctttcacactca |
| | | (SEQ ID NO: 2) |
| Human | Forward | ccactcacctcttcagaacgaa |
| IL-6 | | (SEQ ID NO: 3) |
| | Reverse | ggcaagtctcctcattgaatcc |
| | | (SEQ ID NO: 4) |
| Bovine | Forward | cagcgacactcactcttctacc |
| GAPDH | | (SEQ ID NO: 5) |
| | Reverse | ttactccttggaggccatgt |
| | | (SEQ ID NO: 6) |
| Human | Forward | tggtatcgtggaaggactcatgac |
| GAPDH | | (SEQ ID NO: 7) |
| | Reverse | atgccagtgaggttcccgttcagc |
| | | (SEQ ID NO: 8) |

### C. Evaluation of the Drug Encapsulation Efficiency and Drug Release Behavior of Hydrogel-Forming Polymers

To evaluate the drug encapsulation efficiency of a hydrogel-forming polymer, a test sample containing 10% w/v of a test material (i.e., PN or GN as obtained in the preceding section A or C of the Experimental Procedures of the Synthesis Example, respectively) and pilocarpine nitrate (2% w/v) dissolved in deionized water was prepared at 25° C., followed by injected into a brown colored vial containing 1.5 mL BSS at 34° C. to allow gelation. The drug-incorporated hydrogel thus formed was subsequently transferred to an empty brown colored vial at 25° C., so that the hydrogel was reversed to a sol state. The drug-containing sol contained in said vial at 25° C. was then subjected to high performance liquid chromatography (HPLC) analysis using a L-2400 UV detector, a L-2130 pump (Hitachi, Tokyo, Japan) and a Mightysil RP-18 column (4.6×250 mm, Kanto Chemical, Tokyo, Japan). The mobile phase was a mixture of 5% monobasic potassium phosphate in Milli-Q water (pH adjusted to 2.5 with 85% phosphoric acid)/methanol (85:15 v/v) with a flow rate of 0.7 mL/min. The eluent peak was detected by measuring absorbance at 216 nm. To determine the amount of drug, photometric reading was referenced to a standard curve of peak area versus pilocarpine nitrate concentration (0.1˜500 μg/mL). The drug encapsulation efficiency was calculated as the percentage of pilocarpine nitrate entrapped in the hydrogel, as compared to the initial feeding amount of drug. Results from five independent experiments were averaged.

To evaluate the drug release behavior of a hydrogel-forming polymer, a drug-incorporated hydrogel sample was prepared at 34° C. in the same manner as described above and then transferred to a 34° C. vial containing 1.5 mL of a release buffer (BSS plus 75 ng MMP-2). After incubation at 34° C. with reciprocal shaking (60 rpm) in a thermostatically controlled water bath for a designated time period ranging from 30 min to 2 weeks, 100 μL of the release buffer was collected and analyzed by HPLC, with same volume of fresh release buffer being added into the vial. The amount of pilocarpine nitrate released from the drug-incorporated hydrogel sample was calculated with reference to a standard calibration curve of pilocarpine nitrate. Results from five independent measurements were averaged and further used to calculate the cumulative released amount of pilocarpine nitrate.

### D. Animal Model Test

All animal procedures were approved by the Institutional Review Board and were carried out in accordance with the ARVO Statement for the Use of Animals in Ophthalmic and Vision Research. Thirty adult New Zealand white rabbits (National Laboratory Animal Breeding and Research Center, Taipei, Taiwan, ROC), weighing 3.0-3.5 kg and 16-20 weeks of age, were used for this test. Surgical operation was performed in one eye of each test animal, leaving the other eye as the normal fellow eye. The rabbits were divided into one control and four experimental groups, with n=6 for each group. After glaucoma induction, the rabbits in the four experimental groups received equivalent doses of drug in different formulations, including: topical administration of solution A (Eye Drop group); and for intracameral administration, solution A (Free-Drug group); solution B that contained drug and PN obtained in Section A of the Experimental Procedures of the Synthesis Examples, described supra (PN-Drug group), and solution C that contained drug and GN obtained in Section C of the Experimental Procedures of the Synthesis Examples, described supra (GN-Drug group). Solution A contained 2% w/v pilocarpine nitrate dissolved in deionized water. Solution B contained 2% w/v pilocarpine nitrate and 10% w/v PN dissolved in deionized water. Solution C contained 2% w/v pilocarpine nitrate and 10% w/v GN dissolved in deionized water. The rabbits in the control group (Ctrl) received no drug.

To produce experimental glaucoma in the eyes of rabbits, the applicants adopted a procedure similar to that described in Percicot et al. (1996), J. Pharmacol. Toxicol. Methods, 36(4): 223-228. The rabbits were anesthetized intramuscularly with 2.5 mg/kg body weight of tiletamine hydrochloride/zolazepam hydrochloride mixture (Zoletil; Virbac, Carros, France) and 1 mg/kg body weight of xylazine hydrochloride (Rompun; Bayer, Leverkusen, Germany). 0.1 mg/mL of α-chymotrypsin was injected into the posterior chamber of the rabbit eye using a 30-gauge needle. The tip of the needle was swept across to homogeneously distribute the enzyme throughout the posterior chamber of the rabbit eye, and the needle remained in position for an additional 2 min before withdrawing it, so as to avoid any contact of the enzyme with the corneal endothelium. During the first week of follow-up examination, each operated eye received two drops of tobramycin-dexamethasone ophthalmic solution (Tobradex; Alcon-Couvreur, Puurs, Belgium) and one drop of diclofenac sodium ophthalmic solution (Voltaren; Ciba Vision Ophthalmics, Duluth, Ga., USA) three times a day to prevent eye inflammation and pain. The animals were considered to be glaucomatous when they were examined to have an IOP (intraocular pressure) higher than 30 mmHg in the eye following 4 weeks of α-chymotrypsin injection.

For the Eye Drop group, 50 μL of solution A was topically instilled in the glaucomatous eye. For the Free-Drug, PN-Drug and GN-Drug groups, a 30-gauge needle was injected into the anterior chamber near the limbus to deliver 50 μl of solution A, solution B and solution C, respectively. To determine the therapeutic efficacy of drug, ophthalmic evaluations were performed before and immediately after drug administration. Thereafter, the bilateral eyes of 30 rabbits were examined at predetermined time intervals for 2 weeks.

The morphology of the anterior segment of the rabbit eye, including corneal and lens clarity, the degree of anterior chamber activity, iris and carrier materials were observed by slit-lamp biomicroscopy (Topcon Optical, Tokyo, Japan). The corneal endothelial cell density in the rabbit eye was measured by specular microscopy (Topcon Optical). Each data point is an average of three independent observations.

The IOP was measured using a Schiotz tonometer (AMANN Ophthalmic Instruments, Liptingen, Germany), and calibrated according to the manufacturer's instructions. For each IOP determination, five readings were taken on each eye, alternating the left and right eyes, and the mean value was calculated. The IOP values of the contralateral normal fellow eyes were used as baseline readings. Data were expressed as the difference from baseline values at each time point.

The miosis tests were carried out after acclimatization in a room with constant lighting. The pupil diameter was measured under standardized conditions using a pupillary diameter gauge (Smith and Newphew Pharmaceuticals, Essex, UK). The pupil diameters of glaucomatous eyes were taken as pretreatment baseline values. The results were presented as the average variation of pupillary diameter at each post-drug-administration time point with respect to basal levels from four independent measurements. To evaluate the in vivo drug release, several additional animals of the control and experimental groups were euthanized with CO₂ gas at postoperative 4 hours and 2 weeks. The aqueous humor from each rabbit eye was immediately aspirated using a 30-gauge needle without touching the iris, lens, and corneal endothelium. The concentrations of released pilocarpine nitrate in aqueous humor specimens were analyzed by HPLC as described above. All experiments were performed in sextuplicate.

### Results and Discussion:

### 1. In Vitro Degradation Test

Because of its biodegradability, gelatin was used as backbone networks for grafting non-biodegradable PNIPAAm segments in the study of this invention.

Gelatin is composed primarily of amino acid residues and is susceptible to enzymatic cleavage. Collagenolytic enzymes, including gelatinases, are capable of causing scission of peptide bonds within the characteristic poly-L-proline type of helical regions of the molecule (Elvin Harper et al. (1972), Biopolymers, 11(8):1607-1612). Within the aqueous humor in glaucomatous eyes, the level of MMP-2 (i.e., the predominant gelatinase) is found to be around 50 ng/mL (Marko Määttä et al. (2005), J. Glaucoma, 14(1):64-69). Hence, in vitro carrier degradation by MMP-2 was evaluated in BSS at 34° C.

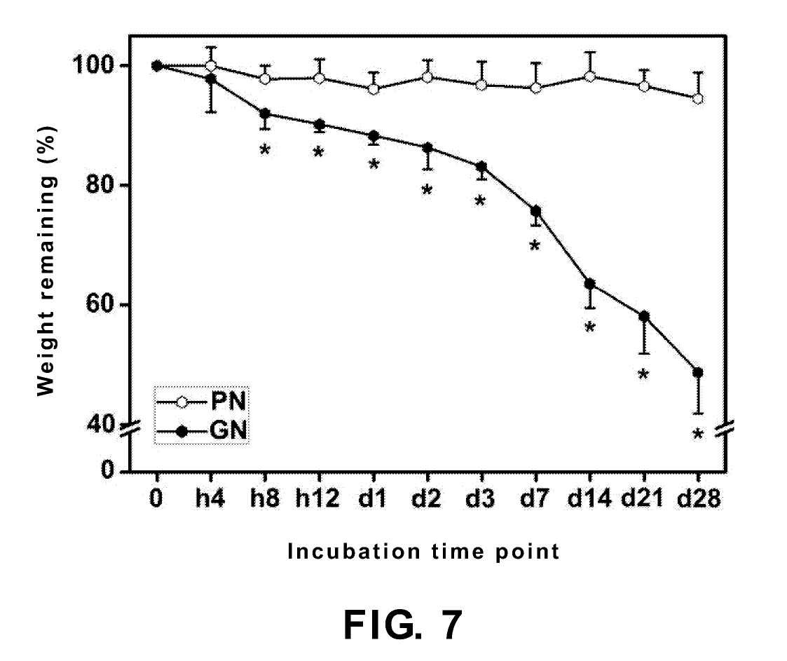

Referring to FIG. 7, the remaining weights of the GN samples were remarkably lower than those of the corresponding PN samples at same incubation time points exceeding 4 hours (P<0.05). During enzymatic degradation time over 28 days, the PN samples appeared to remain original weight (P>0.05). However, the GN samples gradually degraded with time, and approximately 50% of weight loss was observed at the end of the test.

It is reported that under collagenase digestion, the degradation of gelatin is almost complete by 1 hour (Jui-Yang Lai and Ya-Ting Li (2011), Journal of Biomaterials Science, 22:277-295). However, in this invention, significant degradation of GN is noted within the period from 3 to 14 days (P<0.05). The obtained results indicate that the slower degradation rate of GN possibly results from the limited access of enzyme to the active sites in the gelatin peptide sequence by the presence of aggregated PNIPAAm chains.

### 2. In Vitro Biocompatibility Studies

To serve as a carrier material in intraocular DDSs for glaucoma therapy, a biodegradable polymer candidate must be able to form in situ a hydrogel in the anterior chamber of the eye without causing damage to intraocular cells. Therefore, GN and PN were analyzed by in vitro biocompatibility assays using bovine corneal endothelial cell line BCE C/D-1b and human lens epithelial cell line HLE-B3.

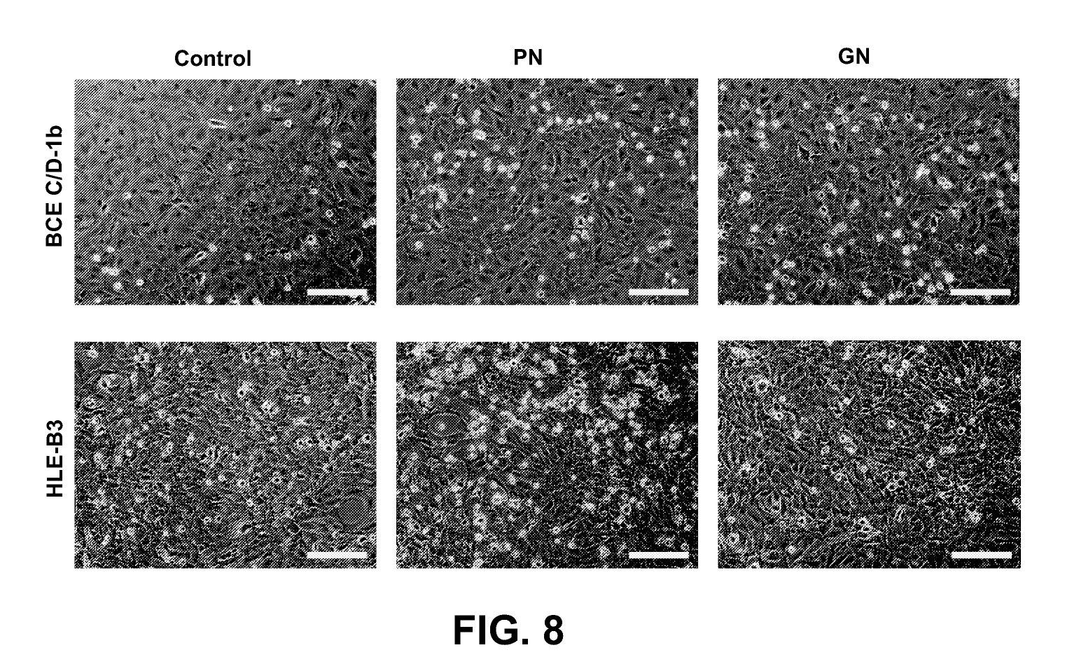

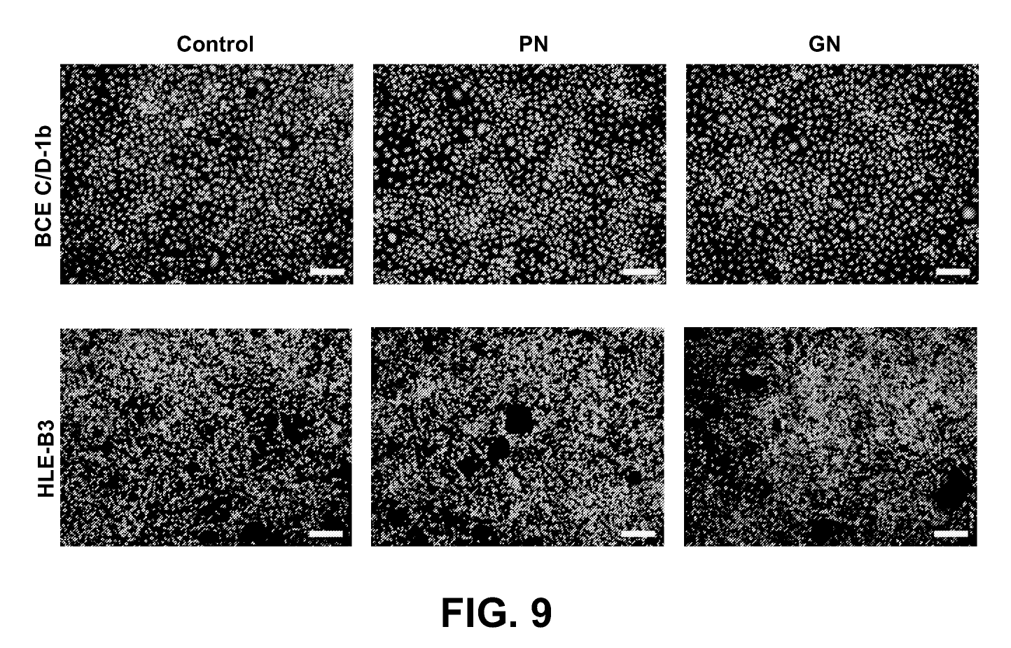

FIG. 8 shows representative images of BCE C/D-1b and HLE-B3 cells photographed after a 2-day exposure to an in situ formed hydrogel from a test material (PN or GN). The cells in the control groups appeared healthy and exhibited typical morphological characteristics. After exposure to the PN and GN samples, the cultured cells actively proliferated and did not reveal any morphological abnormalities. FIG. 9 shows further representative photographs of BCE C/D-1b and HLE-B3 cells assayed by Live/Dead stain, in which the live cells show green fluorescence and dead cells showed red fluorescence. The majority of cultured cells in various test groups were nearly all viable with only a few dead cells, indicating no cytotoxicity of the test samples.

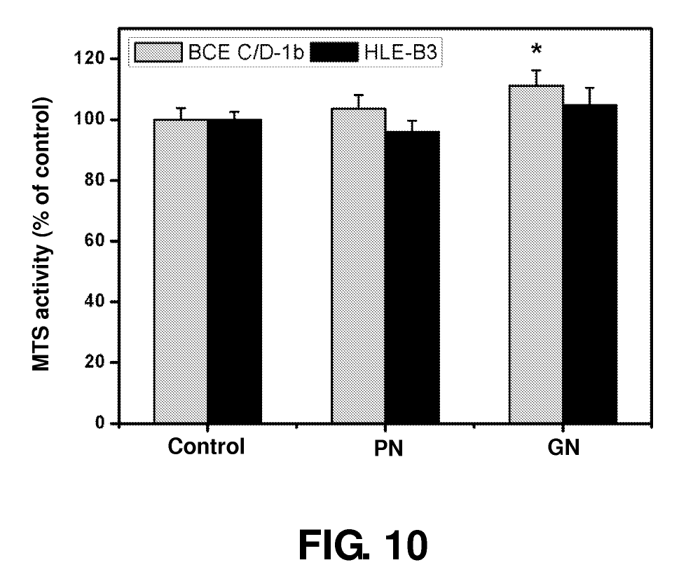

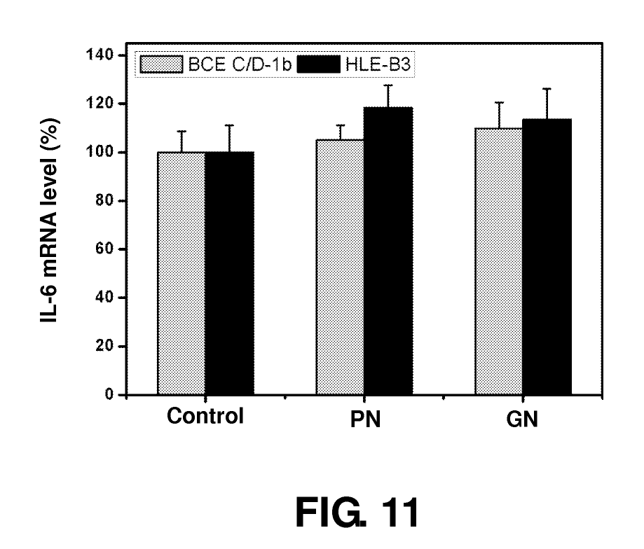

Quantitative analysis for cell growth was further performed using the cell proliferation MTS assays. Referring to FIG. 10, for both BCE C/D-1b and HLE-B3 cells, the mitochondrial dehydrogenase activity (MTS activity) between the PN and GN groups did not show a significant difference (P>0.05), and the detected MTS activities of the PN and GN groups were almost the same with that of the control group. FIG. 11 shows the pro-inflammatory IL-6 gene expression in BCE C/D-1b and HLE-B3 cells after a 2-day exposure to an hydrogel formed in situ from a test material (PN or GN). Similar IL-6 expression levels of BCE C/D-1b and HLE-B3 cells were observed in the control, PN and GN groups and not statistically different (P>0.05). These findings suggest that the culture of anterior segment cells with in situ forming gels does not affect proliferation and has little effect on inflammation.

Investigators have performed biocompatibility studies on thermo-responsive PNIPAAm-based materials. The results, however, are inconsistent. While many reports showed that the PNIPAAm-grafted copolymers had good compatibility towards chondrocytes (Jyh-Ping Chen et al. (2006), Macromol. Biosci., 6:1026-1039) and retinal pigment epithelial cells (Scott D. Fitzpatrick et al. (2010), Biomacromolecules, 11(9):2261-2267, others demonstrated clear cytotoxicity induced by NIPAAm monomers or their polymers in both intestinal Caco-2 and pulmonary Calu-3 cells (Henna Vihola et al. (2005), Biomaterials, 26(16):3055-3064).

According to the applicants' previous study on the control of corneal endothelial cell adhesion/detachment on thermo-responsive culture support, the PNIPAAm coatings are not toxic to living cells (Jui-Yang Lai et al. (2006), Arch Ophthalmol, 124:1441-1448). Here, the PN samples also exhibit non-cytotoxicity for use as drug carriers. On the other hand, the excellent bioaffinity of gelatin makes it an attractive ophthalmic biomaterial (Jui-Yang Lai (2010), J. Mater. Sci.: Mater Med., 21(6):1899-1911). Thus, the combination of PNIPAAm with gelatin may not compromise the safety of graft copolymers manufactured therefrom. This presumption was proved by GN synthesized and tested in this invention.

### 3. In Vitro Drug Release Studies

When the PN or GN solution mixed with pilocarpine was injected into an environment that was maintained at 34° C., the hydrophobic interactions led to the shrinkage of the polymer chains, so that pilocarpine was incorporated (entrapped?) in the in situ formed hydrogel. The HPLC analysis of pilocarpine showed that pilocarpine had a retention time of about 8 min at a flow rate of 0.7 mL/min (data not shown).

The drug encapsulation efficiency of the tested PN and GN samples was found to be 55.0±3.6 and 62.4±2.0% (n=5), respectively, indicating that significantly higher drug payload is related to the faster temperature triggered gelation of the GN copolymers.

According to the synthesis procedures set forth in the preceding section A of Experimental Procedures, the applicants synthesized a second hydrogel-forming polymer using gelatin in place of A-gelatin, and the second hydrogel-forming polymer thus obtained was determined to have a drug encapsulation efficiency of 48.9±1.3 (n=5) in deionized water, which is slightly lower than that of GN.

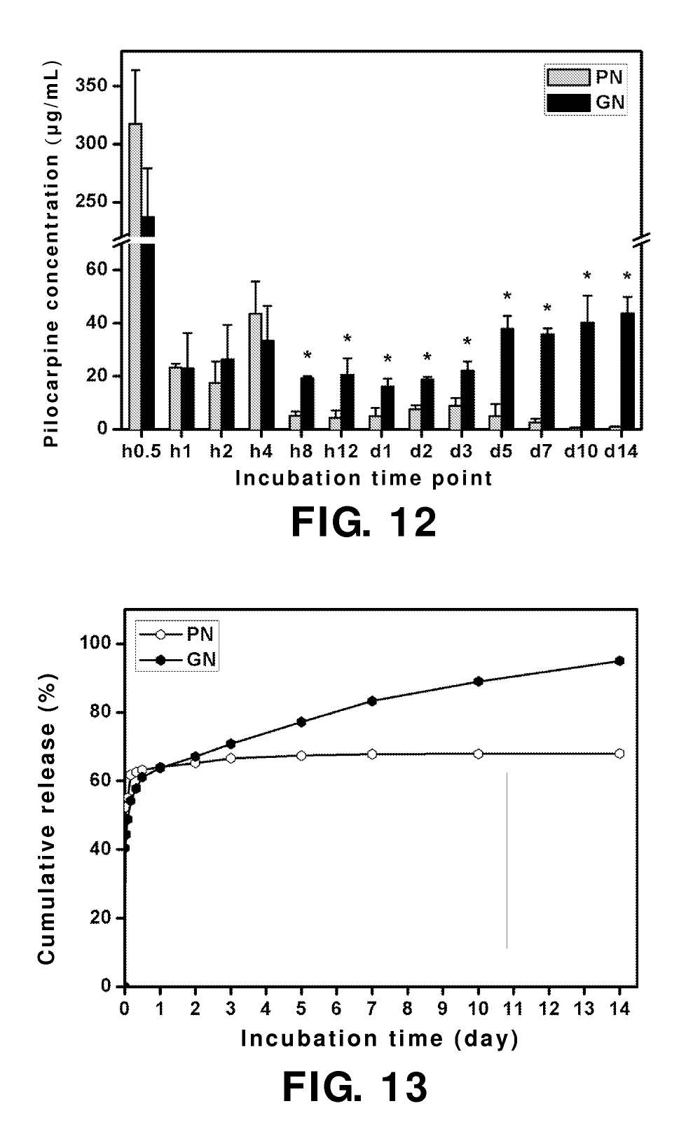

The release profile of pilocarpine from an in situ forming delivery system was studied using PN and GN. FIG. 12 shows the time course of the release of pilocarpine from a pilocarpine-incorporated hydrogel sample prepared from a test material (PN or GN), and FIG. 13 shows the cumulative release profile of pilocarpine as a function of time from said pilocarpine-incorporated hydrogel sample. In all the cases, a burst release was observed at the beginning (i.e., the first 30 min), probably due to desorption of drug initially distributed on the external surface of the carriers. Thereafter, the release rates began to slow down. At each time point from 1 to 4 hours, the pilocarpine concentration did not show any significant difference between the PN and GN groups (P>0.05), suggesting a similar release behavior. After 4 hours, the cumulative drug release in both groups was approximately 60% of the original entrapped amount.

During the period from 8 hours to 14 days, the two test groups were observed to show distinctly different drug release behaviors, which may be attributed to the interactions between the drug and the hydrogel-forming material used. In the PN group, the measured pilocarpine concentration at each sampling time was below 10 mg/mL, and a negligible amount of released drug was detected at 10 and 14 days. For PNIPAAm hydrogels, the dense surface skin formation is known to limit the water outflow from the carrier interior space (Ryo Yoshida et al. (1992), J. Biomater. Sci. Polymer Edn., 3(3):243-252), which may lead to the inhibited release of drug from the in situ formed PN gels. It has been documented that the effective therapeutic dose of pilocarpine for IOP reduction is 10-33 μg/mL in the anterior chamber (Ging-Ho Hsiue et al. (2001), Biomaterials, 22(13):1763-1769). Accordingly, the injectable PN-based DDS tested here seems not suitable for glaucoma treatment. In contrast, in the GN groups, the pilocarpine was continuously released from the tested GN-based DDS, and the cumulative release approached 95% of the original entrapped amount 14 days after injection. It is further noted that the drug concentrations in the release media were sufficient to alleviate the IOP elevation. These results imply that the progressive biodegradation of gelatin is beneficial for the delivery carriers prepared therefrom to provide a sustained release of drugs and an enhanced drug bioavailability.

### 4. Animal Model Test

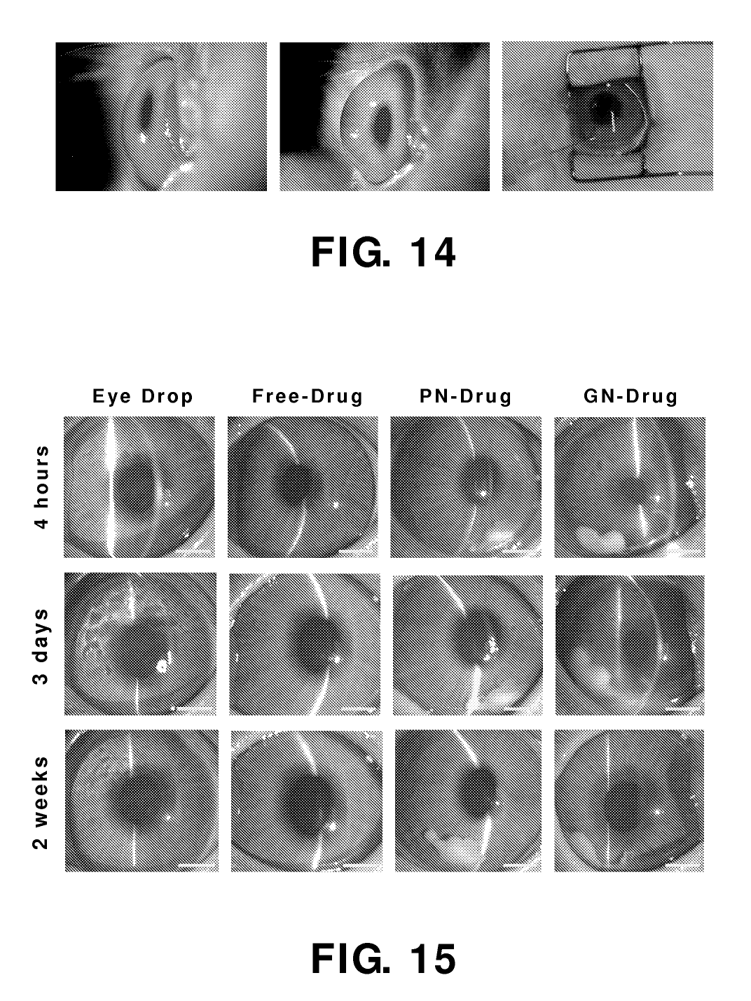

In the study of this invention, chronic ocular hypertension was induced by a single injection of α-chymotrypsin into the posterior chamber of rabbit eyes. Pathogenesis of this experimental glaucoma is attributed to blockage of the trabecular meshwork by lysed zonular material (Kirt N. Gelatt (1977), Invest. Ophthalmol. Vis. Sci., 16(7):592-596). Before operation, the rabbit eye had an anterior chamber of normal depth (FIG. 14, left panel). The α-chymotrypsin injection could obstruct the outflow of aqueous humor sufficiently to cause the increase in IOP (FIG. 14, middle panel). After establishment of animal model, pilocarpine was intracamerally administered using a GN-based DDS made according to this invention (FIG. 14, right panel). When injected directly into the anterior chamber via a 30-gauge needle, the two tested polymer-drug solutions (i.e., solutions B and C) showed an instantaneous phase transition from sol to gel. This finding is in contrast to the results from Section 3.4 showing a longer GFT of GN copolymers characterized by phase transition kinetics.

The therapeutic efficacy of the hydrogel-forming DDSs in glaucomatous eyes was further examined by the following experiments. Slit-lamp biomicroscopy is a powerful tool in anterior segment diagnostics and has previously been used to investigate the biodegradation behavior and biocompatibililty of material implants including amniotic membrane (Pei-Lin Lu et al. (2008), J. Biomed. Mater. Res. A, 86(1):108-116) and hyaluronic acid hydrogel disc (Jui-Yang Lai et al. (2010), J. Biomater. Sci. Polymer Edn., 21(3):359-376) in the applicants' laboratory. FIG. 15 shows the representative slit-lamp biomicroscopic images of rabbit eyes after ocular administration of different dosage forms of pilocarpine. In the Eye Drop and Free-Drug groups, the cornea was clear and the anterior chamber was quiet (no cells or flare) during the follow-up period from 4 hours to 2 weeks, indicating that the topical application of drug solutions or intracameral injection of free drugs does not cause an inflammatory response. For both the PN-Drug and GN-Drug groups, the hydrogels formed in situ after injecting the test solutions into the anterior chamber of the eye between the cornea and iris. At postoperative 3 and 14 days, the in situ formed hydrogel in the PN group remained structurally intact. However, the movement of said hydrogel within the intraocular space was noted, which may result in disturbed physiology and blurred vision. On the other hand, because of their excellent bioadhesion, the in situ formed hydrogel in the GN group had stable residence at the surgical site. With increasing time, the in situ formed hydrogel in the GN group was gradually disintegrated, presumably due to the biodegradation by endogenous MMP-2. Two weeks later, the amount of remnants was around 60% of the original size.

In a previous study, the applicants have shown that after a 14 day incubation with MMP-2, the degradation products of EDC cross-linked gelatin samples have a Mw of ˜4 kDa (Jui-Yang Lai and Ya-Ting Li (2010), Biomacromolecules, 11(5):1387-1397). Given the fact that the aqueous humor leaves the eye by passing through the trabecular meshwork into the canal of Schlemm, the applicants pressumed that the elimination of degraded GN fragments from the eye may occur with the aqueous humor drainage. At the end of the experiment, no cataract, corneal neovascularization, stromal disease or sign of ocular inflammation was observed, suggesting the non-toxicity of the injectable biodegradable GN-based DDS according to this invention.

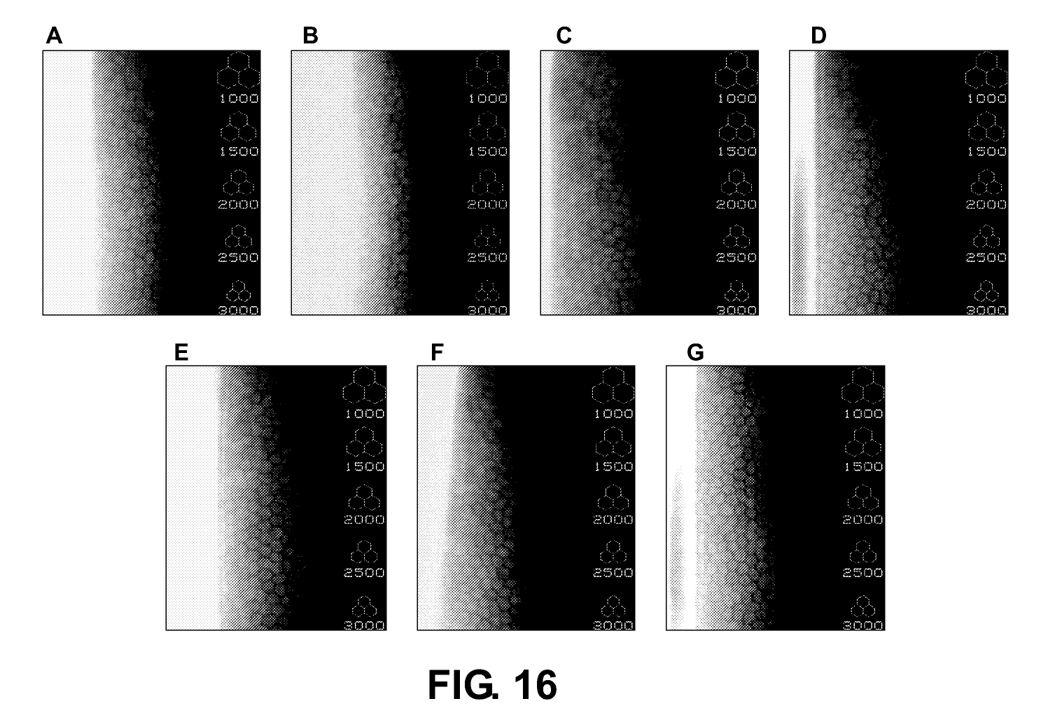

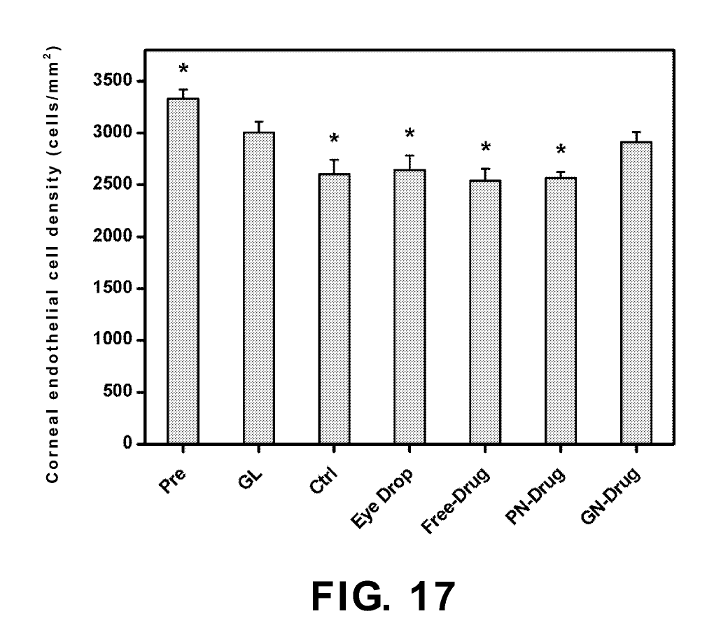

It is crucial to examine the incidence of anterior segment tissue response(s) caused by intracamerally administered drugs due to concerns such as toxicity. The corneal endothelium is a thin cell monolayer that maintains corneal deturgescence and clarity. Once injured, the corneal endothelial cells in vivo demonstrate an irreversible decrease in cell density because of their limited regenerative capacity (Jui-Yang Lai et al. (2006), Arch Ophthalmol., 124(10):1441-1448). Therefore, the change in cell density, which is a sensitive indicator of endothelial damage, was investigated after 2 weeks of pilocarpine administration. Referring to FIGS. 16 and 17 and Table 1, before operation, the normal rabbit corneal endothelial cells on Descemet's membrane packed together and exhibited a typical hexagonal shape. The glaucomatous eyes receiving different ocular dosage forms showed similar morphology. However, the averaged endothelial cell density in the Pre group (rabbit eyes before operation) was 3331±87 cells/mm², which was significantly higher than those found in the GL group (rabbit eyes after glaucoma induction) (3007±103 cells/mm²) (P<0.05).

TABLE 3
Corneal endothelial cell density at different designated time points

| | Corneal endothelial cell density | |
|---|---|---|
| | (cell/mm²) | |
| Before glaucoma induction | 3331 ± 87* | |
| After glaucoma induction | 3007 ± 103 | |
| On Day 14 after drug administration | Ctrl group | 2606 ± 136* |
| | Eye Drop group | 2641 ± 144* |
| | Free Drug group | 2543 ± 111* |
| | PN group | 2568 ± 58* |
| | GN group | 2914 ± 95 |

*p < 0.05 (n = 6) as compared to control group (glaucomatous rabbits receiving no drug).

Cho et al. reported a 13% reduction in corneal endothelial cell count in primary open-angle glaucoma patients as compared to a normal control group (Sung Woo Cho et al. (2009), Jpn. J. Ophthalmol., 53(6):569-573). The results obtained here are compatible with these earlier observations, and indicated that the elevated IOP can significantly induce abnormalities in tissue structure. During follow-up examinations, the cell density in GN-Drug group was maintained at a level of around 2950 cells/mm², while progressive endothelial cell loss was observed in all the other three drug tested groups. These findings suggest that insufficient pharmacological treatment for glaucoma may cause further cell death due to continued high IOP. The preservation of corneal endothelial cell density for the eyes injected with drug-containing GN samples also implies that the released pilocarpine is therapeutically active and efficacious.

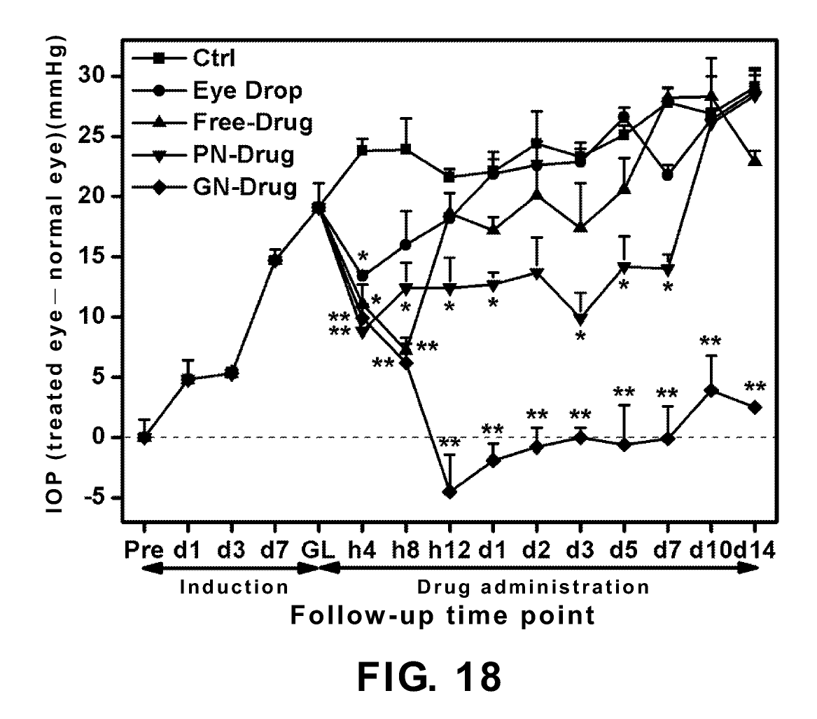

FIG. 18 shows the IOP profile of five different test groups. Mean baseline IOP values ranged from 21.2 to 24.6 mmHg. All glaucomatous eyes had an IOP of around 43 mmHg. In the Ctrl group, the IOP for the eyes without drug treatment was maintained at a high level during the follow-up period of 2 weeks. After ocular administration of different dosage forms of pilocarpine for 4 hours, a remarkable decrease in the IOP was observed (P<0.05). The results could be verified by in vivo drug release studies, which showed that the total pilocarpine concentration in the aqueous humor collected 4 hours after drug administration was 55±10, 103±15, 102±12, and 175±14 mg/mL in the Eye Drop, Free-Drug, PN-Drug, and GN-Drug groups, respectively.

Due to the fast tear washout (15-30 s) and limited corneal penetration (<5%) of topically instilled drugs (Ripal Gaudana et al., (2010), AAPS J, 12(3):348-360), a gradual rise in IOP was then found in the Eye Drop groups. Although intracameral injection of free drugs can achieve longer therapeutic action, the change in the IOP showed a similar tendency, probably caused by drug metabolism and excretion from the eye. At 8 hours after intracameral injection of the drug-containing PN sample, the limited IOP lowering effect might be explained by insufficient amount of released pilocarpine, as mentioned in the preceding section of in vitro drug release studies. In contrast, the IOP in the GN-Drug groups gradually decreased and returned to the baseline values 12 hours after drug administration. Two weeks later, the total pilocarpine concentration in the aqueous humor was almost zero for the Eye Drop, Free-Drug and PN-Drug groups except GN-Drug group (20±8 mg/ml). It was noted that at each time point from 12 hours to 2 weeks, the GN-Drug group significantly differed from all the other three drug tested groups (P<0.05 or P<0.005) with respect to IOP, indicating high ocular bioavailability of pilocarpine. The GN-based DDSs in a sustained release manner at therapeutic concentration may be more effective than other formulations, such as eye drops and injectable suspensions without or with in situ forming PN gels.

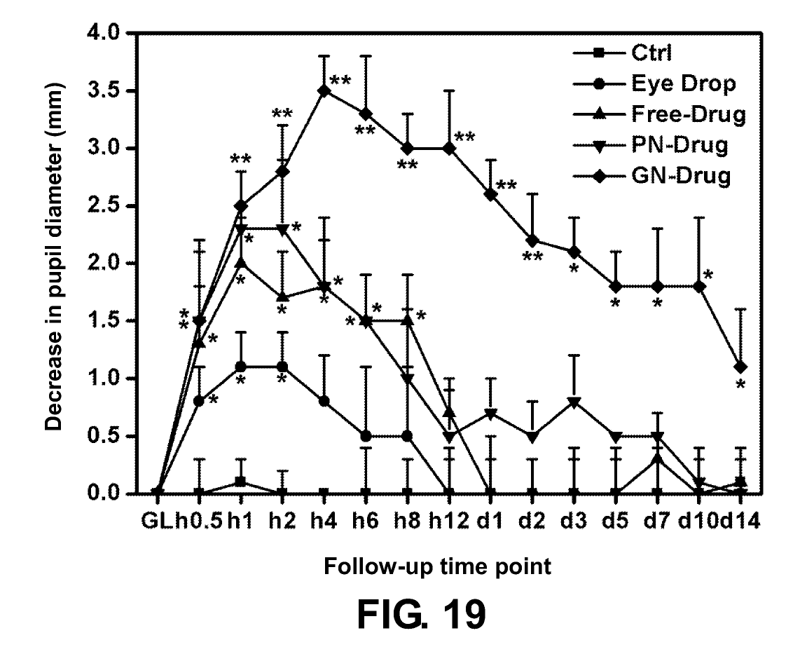

Pilocarpine is a miotic and generally used to treat glaucoma (Ging-Ho Hsiue et at. (2001), Biomaterials, 22(13):1763-1769). FIG. 19 shows the decrease in pupil diameter of rabbits after ocular administration of different dosage forms of pilocarpine. The onset of miosis action was observed within 30 min following topical application of eye drops. It has been reported that the duration of pupillary constriction induced by pilocarpine is usually up to 8 hour (R. Bensinger et al. (1976), Invest Ophthalmol., 15:1008-1010). The experimental results shown in FIG. 19 are compatible with these earlier observations. In the Free-Drug group, the pupil was restored to normal size after 1 day of intracameral injection. For rabbit eyes receiving drug-containing PN sample, an insignificant miotic response was noted over the period from 12 hour to 1 week (P>0.05). However, in the GN-Drug group, the decrease in pupil diameter peaked at 4 hour after drug administration, and the intense pharmacological activity was sustained throughout the entire 2 weeks of the experiment. The results suggest that the miosis action is positively correlated with the IOP lowering effect.

The long-lasting pharmacological profile may reflect the influence of injectable GN carriers on intraocular delivery of pilocarpine.

### CONCLUSIONS

In summary, the applicants have tested the hypothesis that intracameral administration of antiglaucoma medications using biodegradable in situ forming DDSs will give an increase in performance as compared to either eye drop instillation or free drug injection. While drug test samples containing PN as a DDS carrier material demonstrate a limiting drug release pattern, drug test samples containing GN as a DDS carrier material may allow a sustained release of pilocarpine and enhance the ocular drug bioavailability. The strategy based on GN carrier systems helps to improve the effectiveness of glaucoma treatment by extending the pharmacological responses (i.e., miosis and IOP lowering effect and preservation of corneal endothelial cell density) in vivo. These findings indicate that the hydrogel-forming polymer according to this invention may have potential for application as an injectable depot formulation for intraocular drug delivery.

All patents and literature references cited in the present specification as well as the references described therein, are hereby incorporated by reference in their entirety. In case of conflict, the present description, including definitions, will prevail.

While the invention has been described with reference to the above specific embodiments, it is apparent that numerous modifications and variations can be made without departing from the scope and spirit of this invention. It is therefore intended that this invention be limited only as indicated by the appended claim.

We claim:

1. A process of making a hydrogel-forming polymer that forms in situ a biodegradable thermo-responsive hydrogel in an aqueous medium having a physiological temperature, the process comprising:
reacting a biodegradable component having amino functional groups with a thermo-responsive component having a carboxylic acid end group, wherein:
the biodegradable component is a protein selected from the group consisting of natural gelatin, aminated gelatin, natural silk protein, aminated silk protein, or combinations thereof; and
the thermo-responsive component has a lower critical solution temperature lower than the physiological temperature and comprises a reaction product generated from a polymerization reaction of a (meth)acrylamide monomer in the presence of a compound having a carboxyl group in the molecule thereof; and
recovering the hydrogel-forming polymer thus formed.

2. The process of claim 1, wherein in the recovered hydrogel-forming polymer, the thermo-responsive component is coupled to the biodegradable component via a covalent bond formed between the carboxylic acid end group of the thermo-responsive component and one of the amino functional groups of the biodegradable component.

3. The process of claim 1, wherein reacting the biodegradable component with the thermo-responsive component is conducted in the presence of a coupling reagent.

4. The process of claim 3, wherein the coupling reagent is 1-ethyl-3-(3-dimethylaminopropyl)carbodiimide (EDC), N,N′-dicyclohexylcarbodiimide (DCC), N,N′-diisopropylcarbodiimide (DIC), or combinations thereof.

5. The process of claim 1, wherein the biodegradable component has a weight average molecular weight in a range from 10,000 to 300,000.

6. The process of claim 1, wherein the biodegradable component is aminated gelatin formed by aminating natural gelatin with a dihydrazide compound, a diamine compound, or combinations thereof.

7. The process of claim 6, wherein the dihydrazide compound is selected from the group consisting of adipic acid dihydrazide, succinic acid dihydrazide, sebacic acid dihydrazide, valine dihydrazide, isophthalic dihydrazide, carbodihydrazide, icosanedioic acid dihydrazide, or combinations thereof.

8. The process of claim 7, wherein the biodegradable component is aminated gelatin formed by aminating natural gelatin with adipic acid dihydrazide.

9. The process of claim 6, wherein the diamine compound is selected from the group consisting of ethylenediamine, propane-1,3-diamine, butane-1,4-diamine, pentane-1,5-diamine, hexane-1,6-diamine, or combinations thereof.

10. The process of claim 1, wherein the number of amino functional groups in the biodegradable component is in a range from 10 to 200 per mole of the biodegradable component.

11. The process of claim 1, wherein the (meth)acrylamide monomer is N-isopropylacrylamide, N,N-diethylacrylamide, N-n-propylacrylamide, acrylamide, N-isopropylmethacrylamide, methacrylamide, N-methylacrylamide, N-methylmethacrylamide, N-ethylacrylamide, N-ethylmethacrylamide, N-n-propylmethacrylamide, N,N-dimethylmethacrylamide, N,N-dimethylacrylamide, N-n-butylacrylamide, or combinations thereof.

12. The process of claim 11, wherein the (meth)acrylamide monomer is N-isopropylacrylamide.

13. The process of claim 11, wherein the reaction product is a carboxylic end-capped polymer of N-isopropylacrylamide, a carboxylic end-capped polymer of N,N-diethylacrylamide, a carboxylic end-capped copolymer of N-isopropylacrylamide and N,N-diethylacrylamide, or combinations thereof.

14. The process of claim 1, wherein the compound having a carboxyl group in the molecule thereof is a chain transfer agent selected from the group consisting of mercaptoacetic acid, 2-mercaptopropionic acid, 3-mercaptopropionic acid, 4-mercaptobutyric acid, 5-mercaptopentanoic acid, 6-mercaptohexanoic acid, p-mercaptobenzoic acid, 3,3′-dithiodipropionic acid, mercaptosuccinic acid, 5,5′-dithiobis(2-nitrobenzoic acid), 11-mercaptoundecanoic acid, or combinations thereof.

15. The process of claim 14, wherein the chain transfer agent is mercaptoacetic acid.

16. The process of claim 1, wherein the polymerization reaction of the (meth)acrylamide monomer is initiated by irradiation with UV or radioactive rays.

17. The process of claim 1, wherein the (meth)acrylamide monomer is N-isopropylacrylamide, the compound having a carboxyl group in the molecule thereof is mercaptoacetic acid, and the reaction product is a carboxylic end-capped poly(N-isopropylacrylamide) generated from the polymerization reaction of N-isopropylacrylamide in the presence of mercaptoacetic acid.

18. The process of claim 1, wherein the thermo-responsive component has a number average molecular weight in a range from 1,000 to 10,000.

19. A hydrogel-forming polymer made by a process as claimed in any one of claims 1-18.

20. A sustained-release pharmaceutical composition comprising a therapeutic agent, and a hydrogel-forming polymer according to claim 19 as a carrier material.

21. The sustained-release pharmaceutical composition of claim 20, wherein the therapeutic agent is selected from the group consisting of pharmaceutically active compounds, hormones, growth factors, enzymes, DNA, plasmid DNA, RNA, siRNA, viruses, proteins, lipids, pro-inflammatory molecules, antibodies, antibiotics, anti-inflammatory agents, anti-sense nucleotides, transforming nucleic acids, living cells, or combinations thereof.

22. The sustained-release pharmaceutical composition of claim 20, wherein the therapeutic agent is an anesthetic, an analgesic, a dopaminergic antagonist, an anticancer agent, an anti-proliferative agent, an angiogenesis inhibitor, an anti-infective agent, an anti-inflammatory agent, an antiviral agent, an antibiotic, an immunomodulatory agent, a hormone, or combinations thereof.

23. The sustained-release pharmaceutical composition of claim 20, wherein the therapeutic agent is an ophthalmic drug selected from anti-glaucoma agents, anti-cataract agents, dopaminergic antagonists, beta adrenergic blockers, angiogenesis inhibitors, anti-infective agents, antibiotics, antibacterials, antivirals, anti-proliferative agents, anti-allergenics, anti-inflammatory agents, hormonal agents, growth factors, carbonic anhydrase inhibitors, decongestants, miotics, anticholinesterase, mydriatics, sympathomimetics, mucus secretogogue, immunomodulatory agents, mast cell stabilizers, or combinations thereof.

24. The sustained-release pharmaceutical composition of claim 20, wherein the therapeutic agent is an ophthalmic drug selected from the group consisting of pilocarpine, epinephrine, tetracycline, phenylephrine, eserine, phospholine iodide, demecarium bromide, cyclopentolate, homatropine, scopolamine, chlortetracycline, bacitracin, neomycin, polymixin, gramicidin, oxytetracycline, chloramphenicol, gentamycin, penicillin, erythromycin, carbachol, sulfacetamide, polymixin B, idoxuridine, isoflorophate, fluoromethalone, dexamethasone, hydrocortisone, hydrocortisone acetate, dexamethasone 21-phosphate, fluorocinolone, medrysone, prednisolone, methyl prednisolone, prednisolone 21-phosphate, prednisolone acetate, betamethasone, triamcinolone, or combinations thereof.

25. The sustained-release pharmaceutical composition of claim 20, wherein the therapeutic agent is an anti-glaucoma agent selected from the group consisting of pilocarpine, timolol, betaxolol, levobunolol, latanoprost, dorzolamide, epinephrine, dipivalyl epinephrine, brimonidine, or combinations thereof.

26. The sustained-release pharmaceutical composition of claim 20, wherein the composition is manufactured as an injectable liquid.

27. The sustained-release pharmaceutical composition of claim 20, wherein the composition is manufactured into a dosage form for intraocular injection.

28. The sustained-release pharmaceutical composition of claim 20, wherein the composition is manufactured as a dry powder for parenteral administration.

29. The sustained-release pharmaceutical composition of claim 20, wherein the composition has a ratio of the hydrogel-forming polymer to the therapeutic agent in a range from 10:1 to 5:2.

30. A method for treating or preventing an ophthalmic disorder, comprising intraocularly administering into an eye of a mammal in need of such treatment a sustained-release pharmaceutical composition that comprises an ophthalmic drug and a hydrogel-forming polymer according to claim 27.

31. The method of claim 30, wherein the ophthalmic drug is selected from the group consisting of anti-glaucoma agents, anti-cataract agents, dopaminergic antagonists, beta adrenergic blockers, angiogenesis inhibitors, anti-infective agents, antibiotics, antibacterials, antivirals, anti-proliferative agents, anti-allergenics, anti-inflammatory agents, hormonal agents, growth factors, carbonic anhydrase inhibitors, decongestants, miotics, anticholinesterase, mydriatics, sympathomimetics, mucus secretogogue, immunomodulatory agents, mast cell stabilizers, or combinations thereof.

32. The method of claim 30, wherein the sustained-release pharmaceutical composition is administered into the mammal's eye via anterior chamber injection, intra-retinal injection, subretinal injection, intravitreal injection, or suprachoroidal injection.

33. The method of claim 30, wherein the ophthalmic disorder is glaucoma or elevated intraocular pressure.

34. The method of claim 33, wherein the ophthalmic drug is an anti-glaucoma agent selected from the group consisting of pilocarpine, timolol, betaxolol, levobunolol, latanoprost, dorzolamide, epinephrine, dipivalyl epinephrine, brimonidine, or combinations thereof.


SEQUENCE LISTING (8 sequences)

SEQ ID NO: 1 - DNA, 21 bp, Artificial Sequence
  Forward RT-PCR primer for Bovine IL-6 mRNA
ccaccccaggcagactacttc

SEQ ID NO: 2 - DNA, 22 bp, Artificial Sequence
  Reverse RT-PCR primer for Bovine IL-6 mRNA
ctccttgctgctttcacactca

SEQ ID NO: 3 - DNA, 22 bp, Artificial Sequence
  Forward RT-PCR primer for Human IL-6 mRNA
ccactcacctcttcagaacgaa

SEQ ID NO: 4 - DNA, 22 bp, Artificial Sequence
  Reverse RT-PCR primer for Human IL-6 mRNA
ggcaagtctcctcattgaatcc

SEQ ID NO: 5 - DNA, 22 bp, Artificial Sequence
  Forward RT-PCR primer for Bovine GAPDH mRNA
cagcgacactcactcttctacc

SEQ ID NO: 6 - DNA, 20 bp, Artificial Sequence
  Reverse RT-PCR primer for Bovine GAPDH mRNA
ttactccttggaggccatgt

SEQ ID NO: 7 - DNA, 24 bp, Artificial Sequence
  Forward RT-PCR primer for Human GAPDH mRNA
tggtatcgtggaaggactcatgac

SEQ ID NO: 8 - DNA, 24 bp, Artificial Sequence
  Reverse RT-PCR primer for Human GAPDH mRNA
atgccagtgagcttcccgttcagc